(19) United States
(12) Patent Application Publication
(10) Pub. No.: US 2005/0044596 A1
(43) Pub. Date: Feb. 24, 2005

(54) Methods to confer enhanced floral properties to plants

(76) Inventor: Alan G. Smith, Falcon Heights, MN (US)

Correspondence Address: Schwegman, Lundberg, Woessner & Kluth, P.A., P.O. Box 2938, Minneapolis, MN, 55402, US

(21) Appl. No.: 10/804,844

(22) Filed: Mar. 18, 2004

Related U.S. Application Data
(60) Provisional application No. 60/456,280, filed on Mar. 19, 2003.

(51) Int. Cl. C12N 15/82; A01H 1/00; A01H 5/00

(52) U.S. Cl. 800/303; 800/290; 800/287

(57) ABSTRACT

The invention provides methods to confer sterility and enhanced floral properties to plants, and plants having those properties.

### CROSS-REFERENCE TO RELATED APPLICATIONS

The present application claims the benefit of the filing date of U.S. application Ser. No. 60/456,280, filed Mar. 19, 2003, the disclosure of which is incorporated by reference herein.

### STATEMENT OF GOVERNMENT RIGHTS

The invention was made, at least in part, with a grant from the Government of the United States of America (grant 9018737 from the National Science Foundation). The Government may have certain rights to the invention.

### BACKGROUND OF THE INVENTION

Plants with new and altered traits imparted by genetic technologies and recombinant DNA technology in particular are now viewed as the cornerstone of the crop biotechnology industry. Currently, a considerable number of crops plants with novel traits that originated from tissue culture, somatoclonal variation or mutation, as well as genetic engineering, are undergoing field trials and the first stages of commercial release.

Nevertheless, the single largest immediate risk for the use of any plant such as a crop plant with novel traits is the risk of contamination among commercial productions of the same species. Moreover, for perennial plants, the proximity of perennial plants with novel traits to their wild relatives is problematic. For instance, a transgenic tree expressing insect tolerance could cross with a wild species of tree to create a hybrid that expresses insect tolerance. Under managed conditions, e.g., plantations, insect resistance would not have a significant environmental impact. However, as managed conditions are less common than unmanaged conditions, the control of the spread of genes that may carry environmental consequences is a highly desirable goal.

In addition to cross-contamination among commercial crop productions, another concern is the potential spread of crops used as vehicles for producing proteins, e.g., heterologous proteins, of commercial or medicinal value. These novel protein products can potentially contaminate plants destined for food use and export. Although production standards can be implemented that attempt to preserve the identity of individual transgenic lines and reduce unintended contaminations, the outflow of genes to other cultivars will eventually occur.

Attempts have been made to develop methods to specifically remove or identify plants that contain novel traits introduced by recombinant DNA. For example, the use of a conditionally lethal gene, i.e., one which results in plant cell death under certain conditions, has been suggested as a means to selectively kill plant cells containing a specific recombinant DNA (e.g., see WO 94/03619). An example of a conditionally lethal gene is the Agrobacterium Ti plasmid-derived gene encoding the enzyme indole acetamide hydrolase (IAMH). IAMH hydrolyzes indole acetamide, a compound that has essentially no phytohormone activity, to form the active auxin phytohormone indole acetic acid. The enzyme IAMH is capable of hydrolyzing a number of indole amide substrates including naphthalene acetamide, resulting in the production of the well-known synthetic plant growth regulator naphthalene acetic acid (NAA). The application of napthalene acetamide is necessary to discriminate which plants carry the conditionally lethal gene.

Other enzymes may also be used as conditionally lethal genes. These include enzymes which act directly to convert a non-toxic substance to a toxin, such as the enzyme methoxinine dehydrogenase, which converts non-toxic 2-amino-4-methoxy-butanoic acid (methoxinine) to toxic methoxyvinyl glycine (Margraff et al., 1980), the enzyme rhizobitoxine synthase, which converts non-toxic 2-amino-4-methoxy-butanoic acid to toxic 2-amino-4-[2-amino-3-hydroxypropyl]-trans-3-butanoic acid (rhizobitoxine) (Owens et al., 1973), the de-acylase enzyme which acts specifically to convert the inactive herbicide derivative L-N-acetyl-phosphinothricin to the active phytotoxic agent phosphinothricin (EP 617121), and the enzyme phosphonate monoester hydrolase which can hydrolyze inactive ester derivatives of the herbicide glyphosate to form the active herbicide (U.S. Pat. No. 5,254,801). The expression of a conditionally lethal gene that acts on a non-toxic substance to convert the nontoxic substance to a toxic substance is typically regulated by a promoter that is a constitutive promoter expressed in all or most cell types, or a developmentally regulated promoter expressed in certain cell types or at certain stages of development.

Conditionally lethal genes may also be engineered from lethal genes. A lethal gene which is expressed only in response to environmental or physiological conditions is lethal under those conditions. For example, a gene that encodes a lethal activity may be placed under the control of a promoter that is induced in response to, for example, a specific chemical trigger. Nevertheless, the widespread application of chemicals may be impractical and raise additional environmental concerns.

The possibility of using a repressed lethal gene to limit the persistence of hybrid crops has been suggested by Oliver et al. (WO 96/04393). In this system, expression of a lethal gene is blocked by a genetic element that binds a specific repressor protein. The repressor protein is the product of a repressor gene typically of bacterial origin. The genetic element that binds the repressor protein is referred to as a blocking sequence and is constructed such that it further comprises DNA sequences recognized by a DNA recombinase enzyme (e.g., the CRE enzyme). Plants that contain the blocked lethal genes are hybridized with plants comprising the DNA recombinase gene. Either the lethal gene or the recombinase enzyme (or both) is under control of regulatory elements that allow expression only at a specific stage of plant development (e.g., seed embryo). Consequently, the recombinase in the resulting F1 hybrid plant removes the specific blocking sequence and activates the lethal gene so that no F2 plant is produced. Notably, this scheme results in nonviable outcrossed seed, and the method does not apply to self- or open-pollinating varieties.

Another strategy to control the spread of certain germplasm is the use of male- or female-sterile plants. For example, several strategies have been developed for the production of male-sterile plants (Goldberg et al., 1993), including: selective destruction of the tapetum by fusing a ribonuclease gene to a tapetum-specific promoter, TA29 (Mariani et al., 1990); premature dissolution of the callose wall in pollen tetrads by fusing a glucanase gene to the tapetum-specific promoter A9 or Osg6B (Worrall et al., 1992; Tsuchiya et al., 1995); antisense inhibition of flavonoid biosynthesis within tapetal cells (Van der Meer et al., 1992); tapetal-specific expression of the Agrobacterium rhizogenes rolB gene (Spena et al., 1992); and overexpression of the mitochondrial gene atp9 (Hemould et al., 1993).

What is needed is a method to control the spread of germplasm as well as to improve other desirable properties of plants.

### SUMMARY OF THE INVENTION

The invention provides a transformed (transgenic) ornamental plant, the nuclear genome of the cells of which is stably transformed with a recombinant (chimeric) DNA molecule, for instance, a recombinant DNA molecule comprising an expression cassette. The expression cassette comprises a promoter which is capable of directing, for instance, selectively directing, expression of a linked DNA segment in pistil and/or stamen, and a DNA segment encoding a gene product, the expression of which in a plant cell results in the sterility of the plant, e.g., via ablation of female- or male-specific organs or inappropriate expression (temporally and/or spatially) of the gene product. The transformed ornamental plant of the invention is thus a female-sterile and/or male-sterile plant and also comprises flowers with increased longevity and/or an increased number of flowers relative to a corresponding untransformed or nontransgenic plant, i.e., one which does not comprise the expression cassette. Increased flower longevity is a sought-after trait for almost all ornamental plants, including annuals, perennials, herbaceous and woody plants. In one embodiment, the recombinant DNA molecule may also comprise a second promoter which is capable of directing expression of a linked DNA segment in plant cells, and a second DNA segment for a marker gene which, when expressed in cells of the plant, renders those cells or a plant comprising those cells distinguishable and/or separable from other cells or plants which do not contain the marker gene.

Thus, in one embodiment, the invention provides a method to prepare a transformed male-sterile plant having flowers with increased longevity and/or having an increased number of flowers. The method comprises contacting cells of an ornamental plant, e.g., cells which are not from a crop commonly grown on a farm including hay, grain, corn, alfalfa, soybean, cotton or tobacco, e.g., Nicotiana tabacum, with a recombinant DNA molecule comprising an expression cassette comprising a stamen-specific promoter operably linked to DNA segment encoding a gene product, the expression of which in a plant cell results in ablation of that cell, so as to yield transformed plant cells. As used herein, a DNA segment which encodes a gene product, the expression of which in a plant cell results in ablation of that cell, preferably does not encode a gene product that redirects metabolism, e.g., the gene product is not a phytohormone, a receptor therefor, or a gene product of a wild-type Agrobacterium spp. Transformed plant cells are then regenerated to provide a differentiated transformed male-sterile plant comprising an increased number of flowers and/or flowers with increased longevity relative to a corresponding untransformed or nontransgenic plant, i.e., one which does not comprise the expression cassette. Male sterility and the floral properties of the transformed plant are the result of the expression of the gene product encoded by the DNA segment (a “cell ablation DNA”) in the stamen of the differentiated transformed plant.

Preferably, the plant cell employed in the methods of the invention is from a plant where floral senescence is triggered by fertilization or pollination, and preferably, from an ornamental plant. As used herein, a cell of “an ornamental plant” includes a cell of an annual or a perennial plant, including herbaceous and woody plants, and preferably a plant where floral senescence is triggered by fertilization or pollination. In one embodiment a cell of an ornamental plant includes, but is not limited to, a plant cell from Ageratum, Alstroemeria, Alyssum, Anthurium, Antirrhinum, Aronia, Astilbe, Azalea, Begonia, Berberis, Elaegnus, Euonymus, Bleeding-heart, Catharanthus, Celosia, Cephalanthus, Chrysanthemum, Cineraria, Clematis, Coleus, Columbine, Convolvulsus, Coral bells, Coreopsis, Cyclamen, Daffodil, Daylily, Delphinium, Dianthus, Echinacea, Exochorda, Exacum, Elaegnus, Euonymus, Forsythia, Fuchsia, Gaura, Gazania, Geraniums, Gerbera, Gloxinia, Helianthus, Hemerocallis, Hibiscus, Hosta, Iris, Impatiens, Kalanchoe, Lilium, Ligustrem, Lonicera, Rhamnus, Malus, Marigold, Narcissus, New Guinea, Nicotiana, e.g., ornamental tobacco such as N. alata, N. rustica, N. langsdorfii, N. sylvestris, and N. plumbaginifolia, Orchid, e.g., Brassia, Cymbidium, Dendrobium, Doritaenopsis, Phalaenopsis and Cattleya, Pansy/Viola, Pyrus, Peony, Petunia, Phlox, Physocarpus, Primula, Poinsettia, Portulaca, Rhamnus, Rudbeckia, Saintpaulia, Salvia, Sedum, Schlumbergera, Syringa, Tulip, Viola, Verbena, Veronica, Vinca, Zinnia, and Zygocactus. In one embodiment, the plant cell is obtained or isolatable from Ageratum, Alstroemeria, Alyssum, Anthurium, Antirrhinum, Aronia, Astilbe, Azalea, Begonia, Berberis, Elaegnus, Euonymus, Bleeding-heart, Catharanthus, Celosia, Cephalanthus, Chrysanthemum, Cineraria, Clematis, Coleus, Columbine, Convolvulsus, Coral bells, Coreopsis, Cyclamen, Daffodil, Daylily, Delphinium, Dianthus, Echinacea, Exochorda, Exacum, Elaegnus, Euonymus, Forsythia, Fuchsia, Gaura, Gazania, Geraniums, Gerbera, Gloxinia, Helianthus, Hemerocallis, Hibiscus, Hosta, Iris, Impatiens, Kalanchoe, Lilium, Ligustrem, Lonicera, Rhamnus, Malus, Marigold, Narcissus, New Guinea, Nicotiana, e.g., ornamental tobacco such as N. alata N. rustica, N. langsdorfii, N. sylvestris, and N. plumbaginifolia, Orchid, e.g., Brassia, Cymbidium, Dendrobium, Doritaenopsis, Phalaenopsis and Cattleya, Pansy/Viola, Pyrus, Peony, Petunia, Phlox, Physocarpus, Primula, Poinsettia, Portulaca, Rhamnus, Rudbeckia, Saintpaulia, Salvia, Sedum, Schlumbergera, Syringa, Tulip, Viola, Verbena, Veronica, Vinca, Zinnia, and Zygocactus. In another embodiment, the plant cell is obtained or isolatable from Aronia, Berberis, Cephalanthus, Clematis, Exochorda, Elaegnus, Euonymus, Forsythia, Ligustrem, Lonicera, Physocarpus, Rhamnus, and Syringa.

Accordingly, the invention also provides a transformed male-sterile plant having flowers with increased longevity and/or having an increased number of flowers and plants produced by crossing the male-sterile plant or progeny thereof with a male-fertile plant, optionally a male-fertile, female-sterile plant.

As described herein, a promoter isolated from a tomato gene encoding a glycine-rich protein (the 108GRP promoter), was characterized as having expression limited to a specific tissue within the stamen, e.g., the anther. The 108GRP promoter was used to express a barnase open reading frame in tobacco and petunia. The transformed plants were normal except that when anthers became mature, they failed to produce pollen, and, surprisingly, the plants had an increased number of flowers and increased flower longevity. Thus, the use of a stamen-specific promoter to express a cell ablation DNA resulted in plants with flowers that lacked pollen and had enhanced floral properties. Moreover, in the absence of other sources of pollen, the flowers in the male-sterile plants are not fertilized and no seed is produced.

For many important horticultural crops, the production of seeds or fruit is a problem due either to the seeds germinating and acting as a weed or the fruit itself being a detraction. For example, the production of fruit on a crab apple, which is grown primarily for its flowers, can be messy or unattractive when fruit falls on lawns, roads and sidewalks. In the absence of other fertilizing pollen, male-sterile cultivars would not produce seeds or fruit. Thus, male-sterile cultivars prepared by the methods of the invention have highly prized phenotypes, i.e., they are sterile, produce less or no fruit, which likely increases vegetative growth, vigor and productivity, and have increased flower longevity and/or increased flower number. The lack of pollen production by the male-sterile plants of the invention also eliminates gene flow between a cultivated plant and wild or naturally occurring plants, which is an issue when introducing a new cultivar into an environment. Further, as many plants may be invasive due to a large number of seeds produced by the plant, male-sterile plants have reduced invasiveness. Male-sterile cultivars also reduce the number of allergic reactions to pollen in animals sensitive thereto.

In another embodiment, the invention provides a method of preparing transformed female-sterile plants having flowers with increased longevity and/or an increased number of flowers. The method comprises contacting cells of an ornamental plant with a recombinant DNA molecule comprising an expression cassette comprising a pistil-specific promoter operably linked to a DNA segment encoding a gene product, the expression of which in a cell results in ablation of that cell, so as to yield transformed plant cells. The transformed plant cells are regenerated to provide a differentiated transformed female-sterile plant comprising an increased number of flowers and/or flowers with increased longevity relative to a corresponding untransformed or nontransgenic plant, i.e., one which does not comprise the expression cassette. Female sterility and the floral properties of the transformed plant are the result of the expression of the gene product encoded by the DNA segment in the pistil of the differentiated transformed plant.

Further in accordance with this invention are provided a transformed female-sterile plant having flowers with increased longevity and/or an increased number of flowers, and plants produced by crossing the female-sterile plant with a male-sterile plant, e.g., a female-fertile, resulting in a female- and male-sterile plant.

As described herein, a SP41 promoter was characterized as having pistil-specific expression. In particular, the SP41 promoter was characterized as having expression limited to the transmitting tissue within the pistil. When the SP41 promoter was used to express a barnase open reading frame in male-sterile tobacco, the plants were normal except they did not produce seed and, the plants had an increased number of flowers and increased flower longevity. Thus, in the absence of a transmitting tract, the flowers of such a plant can not be fertilized, resulting in the lack of seed production, even in the presence of viable pollen. Moreover, female-sterile cultivars produce less or no fruit. Thus, female-sterile cultivars prepared by the methods of the invention have highly prized phenotypes, i.e., they are sterile, produce less or no seed or fruit even in the presence of pollinators, which likely increases vegetative growth and productivity and decreases invasiveness, and have increased flower longevity and/or increased flower number.

This technology thus has applicability to a diverse group of annual, perennial, woody, and herbaceous horticultural crops. It provides a simple mechanism for enhancing the value of current cultivars for increased flower longevity, increased flower number, decreased invasiveness, reduced gene flow to native populations, elimination of nuisance fruit and seeds, and decreased allergen production. In combination with female-sterility, male-sterility offers the horticulture industry a value added trait.

In another embodiment, the invention provides a method of preparing transformed male- and female-sterile plants having flowers with increased longevity and/or an increased number of flowers. The method comprises crossing a transgenic male-sterile ornamental plant comprising a recombinant DNA molecule comprising an expression cassette comprising a stamen-specific promoter operably linked to a DNA segment encoding a gene product, the expression of which in a cell results in ablation of that cell, with a transgenic female-sterile ornamental plant comprising a recombinant DNA molecule comprising an expression cassette comprising a pistil-specific promoter operably linked to a DNA segment encoding a gene product, the expression of which in a cell results in ablation of that cell, so as to yield a male-sterile, female-sterile progeny plant comprising an increased number of flowers and/or flowers with increased longevity relative to a corresponding untransformed or nontransgenic plant, i.e., one which does not comprise one or both expression cassettes. Sterility and the floral properties of the transformed plant are the result of the expression of the gene product encoded by each DNA segment in the stamen or pistil of the progeny plant.

Further in accordance with this invention are provided a transformed male- and female-sterile plant having flowers with increased longevity and/or an increased number of flowers.

### BRIEF DESCRIPTION OF THE FIGURES

FIG. 1. Schematic of an expression cassette to test the anther specificity of the 108GRP promoter (the 108GRP-GUS expression cassette).

FIG. 2. Cross-section of a tomato anther from a tomato plant carrying the 108GRP-GUS expression cassette. Flowers from a tomato plant carrying 108GRP-GUS were sectioned by hand and placed in a colorless substrate for beta-glucuronidase. After 90 minutes at 37° C., sections were removed from the substrate and decolorized with 95% ethanol.

FIG. 3. Graph of GUS activity in various organs of tomato (A) and tobacco (B) plants containing the 108GRP-GUS expression cassette. (A) The tomato organs assayed for GUS included: whole flowers less than 4 mm in lengths (<4 bud); anthers from flowers that were dissected from flowers 4-5 mm in length (4-5); 5-8 mm in length (5-8); 8-10 mm in length (8-10); 10-12 mm in length (10-12); at anthesis (Anth); flowers 8-11 mm in length with their anthers removed (8-11 F1); roots (Rt); stamens (St); and leaves (Lf). (B) The tobacco organs assayed for GUS included: anthers from flowers that were dissected from flowers 4.5 mm in length (4-5); from flowers 8 mm in length (8); from flowers 11 mm in length (11); from flowers 14 mm in length (14); from flowers 17.5 mm in length (17.5); flowers at anthesis (Anth); flowers 11 mm in length with their anthers removed (11 F1); roots (Rt); stamens (St); and leaves (lf). Significant levels of GUS activity are limited to anthers in tomato or tobacco plants transformed with 108GRP-GUS. Similar conservation was observed in the distantly related plant, Arabidopsis thaliana.

FIG. 4A. Schematic of an expression cassette comprising the 108GRP promoter linked to an open reading frame for barnase (the 108GRP-barnase expression cassette, a “male sterility gene”).

FIG. 4B. Schematic of an expression cassette comprising the SP41 promoter, a pistil-specific promoter, linked to an open reading frame for barnase (SP41-barnase expression cassette, a “female sterility gene”).

FIG. 5. Photograph of tobacco flowers from untransformed tobacco plants (wild-type) and from plants containing the 108GRP-barnase expression cassette and the SP41 promoter-barnase expression cassette. The photographs show the dramatic difference between the tobacco floral organs from plants with barnase expressed in a male- and female-specific manner and the wild-type plant which does not express barnase.

FIG. 6. Photograph showing a greater magnification of the anthers from the wild-type (bottom) and sterile (top) plants shown in FIG. 5. The photos show the dramatic difference between the tobacco anthers from plants with the sterility genes (top) and the wild-type plant (bottom) without these genes.

FIG. 7. Photograph of tobacco stigmas from untransformed tobacco plants (wild-type) and from plants containing the female-sterility gene and the male-sterility gene.

FIG. 8. Photograph of tobacco styles from untransformed tobacco plants (wild-type) and from plants containing the female-sterility gene and the male-sterility gene.

FIG. 9. Micrographs of sections of tobacco styles from untransformed tobacco plants (wild-type) and from plants containing the female-sterility gene and the male-sterility gene.

FIG. 10. Photograph showing an untransformed tobacco plant (WT 8) and a sterile plant containing the 108GRP-barnase expression cassette and the SP41-barnase expression cassette (ST22). The photograph shows that the wild-type and sterile plants have no significant differences in vegetative growth. Thus, the male- and female-sterility genes have phenotypes limited to the anther and pistil only.

FIG. 11. Graph of Lavender Storm (LS) and Purple Wave (PW) petunia flower longevity in plants that express the 108GRP-barnase expression cassette. Flowers were either crossed with non-self pollen (+) or not crossed (−). In this experiment, plants were grown in a greenhouse under optimal conditions and flowers were crossed with non-selfed pollen the day of flower opening. Flower longevity was determined by observing flowers daily and recording the time of corolla senescence.

### DETAILED DESCRIPTION OF THE INVENTION

I. Definitions

“Chimeric” indicates that a DNA sequence, including a vector or a gene, is comprised of more than one DNA sequence of distinct origin, which sequences are fused together by recombinant DNA techniques resulting in a DNA fragment, which does not occur naturally, i.e., in an untransformed or wild-type organism, e.g., plant. Thus, “chimeric” as it relates to a recombinant DNA molecule of the invention means at least one of the components in the expression cassette: 1) is not naturally found under the control of the male- or female-specific promoter; and/or 2) is not naturally found in the same linkage or position. Examples of chimeric DNA sequences include a DNA sequence of bacterial origin which encodes a gene product, the expression of which in a plant cell results in ablation of that cell, under the control of another DNA sequence comprising a male- or female-specific promoter of plant origin; or a DNA sequence of plant origin which encodes a gene product, the expression of which in a plant cell results in ablation of that cell, under the control of another DNA sequence comprising a male- or female-specific promoter of plant origin, where the promoter that is linked to the cell ablation DNA is not naturally linked to that DNA or is linked to that DNA in a different linkage or position than is found in nature.

“Gene” refers to a nucleic acid fragment that expresses mRNA, functional RNA, or specific protein, including regulatory sequences. The term “native gene” refers to gene as found in nature. The term “chimeric gene” refers to any gene that contains 1) DNA sequences, including regulatory and coding sequences, that are not found together in nature, 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes.

The term “endogenous gene” refers to a native gene in its natural location in the genome of an organism. A “foreign” gene refers to a gene not normally found in the host organism but that is introduced by gene transfer.

The terms “protein,” “peptide” and “polypeptide” are used interchangeably herein.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters. However, some suitable regulatory sequences useful in the present invention will include, but are not limited to constitutive plant promoters, plant tissue-specific promoters, plant development-specific promoters, inducible plant promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., 1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., 1989.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

A “minimal or core promoter” is a promoter consisting only of all basal elements needed for transcription initiation, such as a TATA-box and/or initiator.

“Regulated promoter” refers to promoters that direct gene expression not constitutively, but in a temporally- and/or spatially-regulated manner, and include both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. Numerous examples of promoters may be found in the compilation by Okamuro et al., 1989.

A “tissue-specific promoter” refers to a regulated promoter that is not expressed in all plant cells but is primarily, but not necessarily exclusively, expressed in one or a few cell types in specific organs, specific tissues, or specific cell types. A tissue-specific promoter includes a promoter that is temporally regulated.

“Operably linked” refers to the association of nucleic acid sequences on a single nucleic acid fragment so that the function of one is affected by the other. For example, a promoter is operably linked with a coding sequence or functional RNA when it is capable of affecting the expression of that coding sequence or functional RNA (i.e., that the coding sequence or functional RNA is under the transcriptional control of the promoter). Coding sequences can be operably linked to regulatory sequences in sense or antisense orientation. In another example, two coding sequences can be operably linked to each other so as to encode a fusion, e.g., a chimeric, protein.

“Expression” refers to the transcription and stable accumulation of sense (mRNA) or functional RNA. Expression may also refer to the production of protein.

The “expression pattern” of a promoter (with or without enhancer) is the pattern of expression levels which shows where in the plant and in what developmental stage transcription is initiated by said promoter. Expression patterns of a set of promoters are said to be complementary when the expression pattern of one promoter shows little overlap with the expression pattern of the other promoter. The level of expression of a promoter can be determined by measuring the ‘steady state’ concentration of a standard transcribed reporter mRNA. This measurement is indirect since the concentration of the reporter mRNA is dependent not only on its synthesis rate, but also on the rate with which the mRNA is degraded. Therefore the steady state level is the product of synthesis rates and degradation rates.

With respect to expression levels or the tissue-specificity of a promoter, the phrase “substantially similar” means that the levels or specificity (tissue and/or developmental) of expression of the promoter are substantially the same as those of a reference promoter. With respect to an activity of a protein, the phrase “substantially similar” means that the activity of a particular protein is substantially the same as that of a reference protein.

The phrase “substantially similar” with reference to nucleotide and amino acid sequences, includes altered nucleotide sequences can simply reflect the degeneracy of the genetic code but nonetheless encode an amino acid sequence that is identical to a reference amino acid sequence as well as an amino acid sequence for a protein with substantially similar activity to the protein, e.g., one with an overall amino acid identity of 95% or greater relative to the reference sequence. Modifications to the nucleotide or amino acid sequences is well within the routine skill in the art. Moreover, the skilled artisan recognizes that substantially similar nucleotide sequences can also be defined by their ability to hybridize, under stringent conditions, for example, at 65° C. in a solution containing 0.1×SSC and 0.1% sodium dodecyl sulphate (SDS) (prepared from 20×SSC=3.0 M NaCl/0.3 M trisodium citrate and 10% SDS), with a reference nucleotide sequence. Exemplary low stringency conditions include hybridization with a buffer solution of 30 to 35% formamide, 1 M NaCl, 1% SDS at 37° C., and a wash in 1× to 2×SSC at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (Horsch et al., 1985; De Blaere et al., 1987), particle bombardment technology (Klein et al., 1987; U.S. Pat. No. 4,945,050) and vacuum infiltration of Agrobacterium into whole plants (Bechtold et al., 1993). Whole plants may be regenerated from transgenic cells by methods well known to the skilled artisan (see, for example, Fromm et al., 1990).

The terms “transformed,” “transformant,” and “transgenic” refer to plants or plant cells that contain a recombinant DNA molecule integrated into their chromosome. The term “untransformed” refers to normal plants that have not been through the transformation process.

“Stably transformed” refers to cells that have been selected and regenerated on a selection media following transformation.

“Genetically stable” and “heritable” refer to chromosomally-integrated genetic elements that are stably maintained in the plant and stably inherited by progeny through successive generations.

“Wild-type” refers to the normal gene or organism found in nature without any known mutation.

“Genome” refers to the complete genetic material of an organism.

The term “nucleic acid” refers to a polynucleotide which can be single-stranded or double-stranded, composed of monomers (nucleotides) containing a sugar, phosphate and a base which is either a purine or pyrimidine. A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. A “genome” is the entire body of genetic material contained in each cell of an organism. The term “nucleotide sequence” refers to a polymer of DNA or RNA which can be single- or double-stranded, optionally containing synthetic, non-natural or altered nucleotide bases capable of incorporation into DNA or RNA polymers.

The invention encompasses isolated or substantially purified nucleic acid or protein compositions. An “isolated” or “purified” nucleic acid molecule or protein, or fragment thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. Fragments and variants of the disclosed nucleotide sequences and proteins or partial-length proteins encoded thereby are also encompassed by the present invention. By “fragment” is intended a portion of the nucleotide sequence or a portion of the amino acid sequence, which has substantially similar activity relative to the corresponding fill-length polypeptide. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides.

By “variants” is intended substantially similar sequences. For nucleotide sequences, conservative variants include those sequences that, because of the degeneracy of the genetic code, encode the amino acid sequence of a reference polypeptide. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis. Generally, nucleotide sequence variants of the invention will have at least 40, 50, 60, to 70%, generally, 80%, preferably 85%, 90%, up to 95%, 98% sequence identity to the native nucleotide sequence.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Methods for mutagenesis and nucleotide sequence alterations are well known in the art. See, for example, Kunkel, 1985; Kunkel et al., 1987; U.S. Pat. No. 4,873,192; Walker and Gaastra, 1983, and the references cited therein. Guidance as to appropriate amino acid substitutions that do not affect biological activity of the protein of interest may be found in the model of Dayhoff et al. (1978), herein incorporated by reference. Conservative substitutions, such as exchanging one amino acid with another having similar properties, may be preferred. Variant proteins within the scope of the invention are those having one or more modifications which do not substantially alter their activity relative to the activity of the nonvariant protein. Such variants may result from, for example, genetic polymorphism or from human manipulation.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication). Specifically included are shuttle vectors by which is meant a DNA vehicle capable, naturally or by design, of replication in two different host organisms, which may be selected from actinomycetes and related species, bacteria and eucaryotic (e.g. higher plant, mammalian, yeast or fungal cells). Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

A “heterologous DNA sequence” is a DNA sequence not naturally associated with a host cell into which it is introduced, including non-naturally occurring multiple copies of a naturally occurring DNA sequence.

By “flower” is meant to include the entire organ of a flower, as well as one or more of its individual parts such as its shoot axis, sepals, petals, male reproductive organs (or stamens) and female reproductive organs or pistil.

By “female organ” is meant the entire organ of a flower that is involved in the production of female gametes and/or viable seeds and/or viable embryos, as well as one or more of its individual parts such as its ovule, ovary, style, stigma, transmitting tract, vascular tissues, corolla (petal), disc, septum, calyx and placenta tissue.

II. Methods of the Invention

In accordance with this invention, a male-sterile and/or female-sterile plant is produced from an ornamental plant cell by stably transforming the plant cell with a recombinant DNA molecule comprising an expression cassette comprising a promoter capable of selectively directing expression of a linked DNA segment in pistil or stamen, linked to a DNA segment encoding a gene product, the expression of which in a plant cell results in ablation of that cell. In one embodiment, the recombinant DNA molecule comprises a promoter capable of preferentially, and optionally exclusively, expressing a linked DNA segment in stamen. Preferably, the expression cassette also comprises suitable transcription termination signals (including a polyadenylation signal). The expression of the cell ablation DNA in the stamen of the plant renders the plant male-sterile and increases the number and/or lifespan of the flowers of that plant. Preferably, the recombinant DNA molecule also comprises at least one marker DNA that is operably linked to a second promoter and is fused at its 3′ end to suitable transcription termination signals.

In another embodiment, the recombinant DNA molecule comprises a promoter capable of preferentially, and optionally exclusively, expressing a linked DNA segment in pistil. Preferably, the expression cassette further comprises suitable transcription termination signals. The expression of cell ablation DNA in the pistil of the plant renders the plant female-sterile and increases the number and/or lifespan of the flowers of that plant. Preferably, a recombinant DNA molecule comprising such as expression cassette includes at least one marker DNA that is operably linked to a second promoter and is fused at its 3′ end to suitable transcription termination signals.

The cell of a plant (particularly a plant capable of being infected with Agrobacterium) may be transformed in accordance with this invention by any method, e.g., using a vector that is a disarmed Ti-plasmid containing the expression cassette and carried by Agrobacterium. Such a transformation can be carried out using procedures described, for example, in EPA 116,718 and EPA 270,822. Preferred Ti-plasmid vectors contain the expression cassette between the border sequences, or at least located to the left of the right border sequence, of the T-DNA of the Ti-plasmid. Other types of vectors or methods can be used to transform the plant cell, using procedures including but not limited to direct gene transfer (as described, for example, in EPA 223,247), pollen-mediated transformation (as described, for example, in EPA 270,356, WO 85/01856, and EPA 275,069), in vitro protoplast transformation (as described, for example, in U.S. Pat. No. 4,684,611), plant RNA virus-mediated transformation (as described, for example, in EPA 067,553, and U.S. Pat. No. 4,407,956) and liposome-mediated transformation (as described, for example, in U.S. Pat. No. 4,536,475).

In one embodiment, a nuclear male-sterile and/or female-sterile plant of the invention is provided by transforming a plant cell with a disarmed Ti-plasmid vector containing the expression cassette and a marker DNA under the control of a second promoter. The marker DNA can be upstream or downstream of the expression cassette in the Ti-plasmid vector, but preferably, the two are adjacent to one another and are located between the border sequences or at least located to the left of the right border sequence of the Ti-plasmid vector, so that they are properly transferred together into the nuclear genome of the plant cell.

A. The Recombinant DNA Molecule

A recombinant DNA molecule of the invention comprises an expression cassette comprising a promoter which is selectively expressed in male- or female-specific tissues. Thus, the expression cassette comprises a transcriptional initiation region, e.g., a male- or female-specific promoter, linked to a DNA segment encoding a gene product, the expression of which in a plant cell results in ablation of that cell. The recombinant DNA molecule may include a plurality of restriction sites for insertion of DNAs of interest including the transcriptional regulatory elements and one or more DNA segments encoding a cell ablating gene product. The recombinant DNA molecule may additionally contain one or more selectable marker genes.

The expression cassette will generally include in the 5′-3′ direction of transcription, a transcriptional and translational initiation region, a DNA segment of interest, and a transcriptional and translational termination region functional in plants. The termination region may be native with the transcriptional initiation region, may be native with the DNA segment of interest, or may be derived from another source. Convenient termination regions are available from the Ti-plasmid of A. tumefaciens, such as the octopine synthase and nopaline synthase termination regions. See also, Guerineau et al., 1991; Proudfoot, 1991; Sanfacon et al., 1991; Mogen et al., 1990; Munroe et al., 1990; Ballas et al., 1989; Joshi et al., 1987.

The selection of an appropriate expression vector for the recombinant DNA molecule will depend upon the method of introducing the expression vector into host cells. One type of an expression vector contains (1) prokaryotic DNA elements coding for a bacterial replication origin and an antibiotic resistance gene to provide for the amplification and selection of the expression vector in a bacterial host; (2) DNA elements that control initiation of transcription such as a promoter; (3) DNA elements that control the processing of transcripts such as introns, transcription termination/polyadenylation sequence; and (4) a reporter gene that is operatively linked to the DNA elements to control transcription initiation. Useful reporter genes include beta-glucuronidase, beta-galactosidase, chloramphenicol acetyl transferase, luciferase, green fluorescent protein (GFP) and the like. The general descriptions of plant expression vectors and reporter genes can be found in Gruber et al. (1993).

1. DNA Segments Useful in the Invention

The selection of the DNA segment which encodes a gene that, when present in the cells of a plant, results in sterility of that plant, e.g., a DNA segment which encodes a cell ablating gene product, is not critical. In one embodiment, a suitable DNA segment encodes a gene product which significantly disturbs the development of stamen or pistil in which the DNA segment is expressed, preferably so as to result in ablation of stamen and/or pistil. “Ablation” of a cell or tissue as used herein means the absence (lack of development) of that cell or tissue in a plant, which absence is associated with the presence of a DNA segment of the invention in that plant, i.e., relative to a corresponding plant which does not comprise and express the DNA segment. In some embodiments of the invention, the inappropriate expression of a DNA segment in a plant results in a cell or tissue which lacks or has reduced function relative to a corresponding plant which does not contain and express the DNA segment. In some embodiments, the suitable DNA segment may be operably linked to a stamen- or pistil-specific promoter, while in other embodiments, e.g., when the DNA segment is a gene essential for floral organ development, a non-tissue specific promoter may be employed. Examples of suitable DNA segments for use in the invention are those which encode a RNase such as RNase T1 (which degrades RNA molecules by hydrolyzing the bond after any guanine residue), T₂ (Kawata et al., 1988), Rh (Horiuchi et al., 1988), or barnase; a DNase such as an endonuclease (e.g., EcoRI); or a protease such as a papain (e.g., papain zymogen and papain active protein). Other examples of suitable DNA segments which encode a cell ablating gene product include, but are not limited to, a lipase such as phospholipase A₂ (Verheij et al., 1981); a lipid peroxidase; a plant cell wall inhibitor; a toxin such as a bacterial toxin (e.g., the A-fragment of diphtheria toxin or botulin); a glycoprotein such as those encoded by the S1, S2, S3, S6 and S7 alleles, particularly of Nicotiana alata (McClure et al., 1989); ribosome inhibitor protein; ricin or alpha-sarain (Larsson et al., 2002); QM proteins (U.S. Pat. No. 5,583,210); callase (WO 93/02197); a gene product for cytoplasmic male sterility (U.S. Pat. No. 5,530,191); TURF 13 (Braun et al., 1990); Gin recombinase (Plasterk et al., 1983; Maeser et al., 1991); CytA toxin (U.S. Pat. No. 4,918,006); adenine phosphoribosyl transferase (Moffatt et al., 1985); gene products that alter plant hormone levels or balance, e.g., rolB (Spena et al., 1992) or indoleacetic acid-lysine synthetase (Spena et al., 1991); or salicylate hydroxylase (Friederich et al., 1995). In one embodiment, the DNA segment does not encode a phytohormone, such as isopentenyl transferase, or a gene product of wild-type Agrobacterium. In another embodiment, the DNA segment does not encode a gene product that alters plant hormone levels or balance.

DNA segments comprising a gene, e.g., a homeotic gene or other regulatory gene, for male or female organ development such as apetal-2, or a gene for hormone synthesis, which hormone is required for fertile flower development, or a portion thereof, may be employed to prepare constructs useful to knock out, e.g., via homologous recombination, expression of that gene, resulting in a sterile phenotype as well as in expression cassettes that result in aberrant or inappropriate expression of that gene, e.g., constitutive or overexpression, thus leading to a sterile phenotype. Examples of homeotic genes are described in Mandel et al. (1992); Yanofsky et al. (1998) (the agamous gene); U.S. Pat. No. 6,444,877 (LSAG gene); Jack et al. (1992) (the apetala3 or pistilata gene); Trobner et al. (1992) (the globosa or deficiens gene); Bauman et al. (1992); Mandel et al. (1995) (the superman gene); Weigel et al. (1992) (the leafy gene); Coln et al. (1991) (the floricaula gene); and Hamilton et al. (1989).

Another example of a suitable DNA segment is an antisense DNA which encodes a strand of DNA complementary to a strand of DNA (the “target” gene) that is only transcribed in the stamen or pistil, e.g., a pistil-specific antisense DNA under the control of a pistil-specific or a constitutive promoter, or complementary to a homeotic gene, e.g., one under control of a stamen- or pistil-specific promoter. Such an antisense DNA can be transcribed into an RNA sequence capable of binding to the coding and/or non-coding portion of an RNA naturally produced in the stamen or pistil, so as to inhibit the translation of the naturally produced RNA. An example of such an antisense DNA for male sterility is the antisense DNA of the TA29 gene which is naturally expressed under the control of the TA29 promoter in tapetum cells of the anthers of plants or an antisense DNA of the gene described in Mascarenhas et al. (1975). Examples of such an antisense DNA for female sterility are antisense DNAs of: the STMG-type genes, such as the STMG07 gene, the STMG08 gene, the STMG4B12 gene and the STMG3C9 gene; the KTI3 gene (Jofuku and Goldberg, 1989); a gene encoding a seed-specific storage protein, such as a 2S albumin (Krebbers et al., 1988; Altenbach et al., 1987; Scolfield and Crouch, 1987); the taz1 gene (see, e.g., Kapoor et al., 2002); a gene corresponding to the cDNA in clone pMON9608, pMON9601, pMON9604, pMON9606, pMON9611, pMON9612, pMON9614, pMON9617, and pMON9619 (Gasser et al., 1989); the SP41 gene (Ori et al., 1990; Sessa et al., 1995); the Sin1 gene (Lang et al., 1994); the pistil-specific gene in U.S. Pat. No. 5,859,328 (see, e.g., SEQ ID NO:9); the ovule-specific gene described in U.S. Pat. No. 5,907,082 (see, e.g., SEQ ID NO:11); and the STIG1 gene (Goldman et al., 1994).

A yet further example of a DNA segment of the invention is one which encodes a specific RNA enzyme (i.e., a so-called “ribozyme”), capable of highly specific cleavage against a given target sequence, as described by Haseloff and Gerlach (1988). The ribozyme is complementary to a strand of DNA (the “target” gene) that is only transcribed in the stamen or pistil, e.g., one under the control of a constitutive promoter, or is complementary to a homeotic gene, e.g., it is under the control of a stamen- or pistil-specific promoter. Such a ribozyme for male sterility is, for example, a ribozyme targeted against the RNA encoded by the TA29 gene, the gene described in Mascarenhas et al. (1975), or a homeotic gene which controls male organ development. For female sterility, such a ribozyme is, for instance a ribozyme targeted against RNA encoded by STMG07 gene, the STMG08 gene, the STMG4B12 gene, the STMG3C9 gene, the KTI3 gene, a gene encoding a seed-specific storage protein such as a 2S albumin, the taz1 gene, or the gene corresponding to the cDNA in clone pMON9608, pMON9601, pMON9604, pMON9606, pMON9611, pMON9612, pMON9614, pMON9617, and pMON9619 (Gasser et al., 1989), the SP41 gene (Ori et al., 1990; Sessa et al., 1995), the Sin1 gene (Lang et al., 1994), the pistil-specific gene in U.S. Pat. No. 5,859,328 (see, e.g., SEQ ID NO:9), the ovule-specific gene described in U.S. Pat. No. 5,907,082 (see, e.g., SEQ ID NO:11), the STIG1 gene (Goldman et al., 1994), or a homeotic gene which controls female organ development.

Preferred DNA segments for use in the invention encode gene products which degrade cellular components, e.g., nucleases, lipases, proteases and glycosylases, or inhibit macromolecular synthesis, e.g., inhibit transcription, translation or replication, i.e., they are general inhibitors of cellular function, and include but are not limited to a DNA segment which encodes a DNase, a RNase, e.g., RNase T1 and barnase, a protease, e.g., pectate lyase, or a toxin, e.g., diphtheria toxin A.

2. Promoters and Other Transcriptional Regulatory Sequences

Within a plant promoter region there are several domains that are necessary for full function of the promoter. The first of these domains lies immediately upstream of the structural gene and forms the “core promoter region” containing consensus sequences, normally 70 base pairs immediately upstream of the gene. The core promoter region contains the characteristic CAAT and TATA boxes plus surrounding sequences, and represents a transcription initiation sequence that defines the transcription start point for the structural gene. The precise length of the core promoter region is indefinite.

The presence of the core promoter region defines a sequence as being a promoter: if the region is absent, the promoter is non-functional. Furthermore, the core promoter region is insufficient to provide full promoter activity. A series of regulatory sequences upstream of the core constitute the remainder of the promoter. The regulatory sequences determine expression level, the spatial and temporal pattern of expression and, for an important subset of promoters, expression under inductive conditions (regulation by external factors such as light, temperature, chemicals, hormones).

Obtaining sufficient levels of transgene expression in the appropriate plant tissues is an important aspect in the production of genetically engineered plants. Expression of a transgene in a plant host is dependent upon the presence of an operably linked promoter that is functional within the plant host. Choice of the promoter sequence will determine when and where within the organism the recombinant DNA molecule is expressed.

Where expression in specific tissues or organs is desired, tissue-specific promoters may be used. In contrast, where gene expression in response to a stimulus is desired, inducible promoters are the regulatory elements of choice. Where continuous expression is desired throughout the cells of a plant, constitutive promoters are utilized. Additional regulatory sequences upstream and/or downstream from the core promoter sequence may be included in expression constructs of transformation vectors to bring about varying levels of expression of heterologous nucleotide sequences in a transgenic plant.

In addition to the use of a particular promoter, other types of elements can influence expression of transgenes. In particular, introns have demonstrated the potential for enhancing transgene expression. For example, Callis et al., (1987) described an intron from the corn alcohol dehydrogenase gene, which is capable of enhancing the expression of transgenes in transgenic plant cells. Similarly, Vasil et al. (1989) described an intron from the corn sucrose synthase gene having similar enhancing activity. The rice actin 1 intron, has been widely used in the enhancement of transgene expression in a number of different transgenic plants (McElroy et al., 1991).

In accordance with the present invention, expression cassette are provided that allow initiation of transcription in a “tissue-specific,” manner. By “tissue-specific,” is intended preferential; or preferably exclusive, expression in cells of a particular tissue. Some variation in absolute levels of expression can exist among different tissues or stages.

Promoters useful in the recombinant DNA molecule of the invention are specifically expressed in either stamen, e.g., anther, pollen, filament or tapetum (male-specific tissue) or pistil, e.g., stigma, ovary, ovule and the transmitting tract (female-specific tissue).

Stamen- and pistil-specific promoter sequences may be obtained from any plant species by methods known to the art, and subsequently used as probes to screen for homologous promoters in other plants by hybridization under low stringency conditions. Alternatively, regions of the stamen- and pistil-specific promoter sequences which are conserved among species can be used as PCR primers to amplify a longer segment and that longer segment used as a hybridization probe (the latter approach permitting higher stringency screening).

A “stamen-specific promoter” (by “stamen” is meant the organ of the flower that produces the male gamete and that includes an anther and a filament) can be an endogenous promoter or an exogenous promoter and can be from the nuclear genome or from the mitochondrial or chloroplast genome of a plant cell. Preferably, the promoter causes the linked DNA segment to be expressed only in anther, pollen or filament cells, for instance in tapetum or anther epidermal cells. The first promoter can be selected and isolated in a well-known manner from any species of plant so long as the promoter directs expression of the DNA which encodes a gene product, the expression of which in a plant cell results in ablation of that cell, specifically in stamen and preferably not in other parts of the plant. For example, a suitable stamen-specific promoter can be identified and isolated by identifying an mRNA which is only present in the plant during the development of its stamen, preferably its anthers, pollen or filament, isolating the stamen-specific mRNA and preparing a cDNA therefrom, using the cDNA as a probe to identify the regions in the plant genome which contain DNA coding for the stamen-specific mRNA; and identifying the portion of the plant genome that is upstream (i.e., 5′) from the DNA coding for the stamen-specific mRNA and that contains the promoter of this DNA. Alternatively, once the sequence of a stamen-specific mRNA is determined, genomic plant databases may be searched to identify the 5′ region.

Examples of such promoters include, but are not limited to, the 108GRP promoter, which was isolated from tomato and is anther-specific, and the TA29 promoter, the TA26 promoter and the TA13 promoter, which were isolated from tobacco and are tapetum-specific. Other male-specific promoters include promoter of a CHSa gene (Meer et al., 1990); CHI gene (Tunen et al., 1989); Bp10 gene (Albani et al., 1992); as well as those described in WO 99/42587, JP2001145430, WO 01/12799 (see, e.g., SEQ ID NO:10 therein); U.S. Pat. No. 5,545,546 (see, e.g., SEQ ID NO:18 therein), U.S. Pat. No. 5,412,085 (see, e.g., SEQ ID NO:8 therein), WO 02/057470 (see, e.g., SEQ ID NO:3 therein), the LAT59 gene (Twell et al., 1991); a promoter for the LAT56 gene (Twell et al., 1991); the taz1 gene (Kapoor et al., 2002); pine cone promoters disclosed in AF337658; WO 01/0188167 (see, e.g., SEQ ID NO:1 therein); U.S. Pat. No. 5,859,328 (see, for instance, SEQ ID NO:9 therein); the rice anther-specific promoter disclosed in Jeon et al. (1991); the E1 gene (WO 92/13950); the anther-specific promoter 5126 (see, for instance, SEQ ID NO:23 in U.S. Pat. No. 5,689,049); the promoter of the gene described in Mascarenhas et al. (1975), the adp1 promoter (Xu et al., 1993), and the pollen-specific SF3 promoter (U.S. Pat. No. 6,452,069). Preferred anther-specific promoters include, but are not limited to, a promoter from a beta-1,3,-glucanase gene or a calcium/calmodulin-dependent protein kinase gene (see U.S. Pat. No. 6,362,395), or a A6, A3, A9, OsgGB, TA29, TA13 (see U.S. Pat. No. 5,652,354), 108, or 5126 or TA39 promoter (see U.S. Pat. No. 6,281,348). Any male-specific gene can be used as a probe to isolate a homologous gene, and therefore a male-specific promoter, from the genome of another plant species. Under hybridizing conditions, such a probe will hybridize to DNA coding for a male-specific mRNA in a mixture of DNA sequences from the genome of the other plant species (Sambrook et al., Molecular Cloning. A Laboratory Manual. Ed. Cold Spring Harbor Laboratory, 1989).

A pistil-specific promoter can be an endogenous promoter or an exogenous promoter and can be from the nuclear genome or from the mitochondrial or chloroplast genome of a plant cell. Preferably, the promoter causes a linked DNA segment to be expressed only in pistil, and preferably in the transmitting tract. The promoter can be selected and isolated in a well-known manner from any species of plant, so long as the promoter directs expression of the linked DNA. The promoter is preferably also selected and isolated so that it does not express the linked DNA segment in other parts of the plant. For example, a suitable pistil-specific promoter can be identified and isolated by identifying an mRNA which is only expressed in pistil, isolating the pistil-specific mRNA and preparing a cDNA therefrom, using the cDNA as a probe to identify the regions in the plant genome which contain DNA coding for this specific mRNA, and identifying the portion of the plant genome that is upstream (i.e., 5′) from the DNA coding for this specific mRNA and that contains the promoter of this DNA. Alternatively, once the sequence of a pistil-specific mRNA is determined, genomic plant databases may be searched to identify the 5′ region.

Examples of such a promoter include, but are not limited to, the SP41 promoter, which is transmitting tract-specific, the Nicotiana tabacum promoters of STMG-type genes, which are style and/or stigma specific promoters, a promoter disclosed in U.S. Pat. No. 5,859,328, a promoter from the S-locus glycoprotein gene, e.g., SLG13, the S-locus related genes, as well as promoters from the STMG-type genes, such as the STMG07 gene, the STMG08 gene, the STMG4B12 gene and the STMG3C9 gene; the KTI3 gene (Jofuku and Goldberg, 1989); a gene encoding a seed-specific storage protein, such as a 2S albumin (Krebbers et al., 1988; Altenbach et al., 1987; Scolfield and Crouch, 1987); the taz1 gene (see, e.g., Kapoor et al., 2002); a gene corresponding to the cDNA in clone pMON9608, pMON9601, pMON9604, pMON9606, pMON9611, pMON9612, pMON9614, pMON9617, and pMON9619 (Gasser et al., 1989); the SP41 gene (Ori et al., 1990; Sessa et al., 1995); the Sin1 gene (Lang et al., 1994); the pistil-specific gene in U.S. Pat. No. 5,859,328 (see, e.g., SEQ ID NO:9); the ovule-specific gene described in U.S. Pat. No. 5,907,082 (see, e.g., SEQ ID NO:11); and the STIG1 gene (Goldman et al., 1994). Other stigma- or transmitting tract-specific promoters from other plant species can be isolated from their genomes, using, for example, any female-specific gene. Under hybridizing conditions, such a probe will hybridize to DNA coding, for example, a stigma- or transmitting tract-specific, mRNA in a mixture of DNA sequences from the genome of the other plant species (Sambrook et al., Molecular Cloning. A Laboratory Manual. Ed. Cold Spring Harbor Laboratory (1989)). Thereafter, another pistil-, stigma- or transmitting tract-specific promoter can be identified. Other style-specific promotors can be isolated from self-incompatibility genes, such as an S-gene, for example as isolated from Nicotiana alata (McClure et al., 1989). Other female organ-specific promoters can be identified using other female organ-specific cDNAs, such as cDNA clone pMON9608 (Gasser et al., 1989) that hybridizes exclusively with a gene expressed only in the ovules of tomato plants.

Moreover, synthetic stamen-specific and/or pistil-specific promoters may also be employed in the expression cassettes of the invention.

Promoters that are capable of driving the expression of a coding sequence in a target cell or tissue, i.e., in stamen or pistil, can optionally be used with combinations of other elements from the 5′-flanking regions of plant, e.g., enhancer, upstream elements, and/or activating sequences, as well as 3′ untranslated elements.

In the recombinant DNA molecule, 3′ transcription regulation signals can be selected among those which are capable of enabling correct transcription termination and polyadenylation of mRNA in plant cells. The transcription regulation signals can be the natural ones of the gene to be transcribed but can also be foreign or heterologous. Examples of heterologous transcription regulation signals are those of the octopine synthase gene (Gielen et al., 1984) and the T-DNA gene 7 (Velten and Schell, 1985).

3. Marker Genes

The selection of a marker DNA is not critical. A suitable marker DNA can be selected and isolated in a well known manner, so that it encodes a RNA or protein that allows plant cells and plants, expressing the marker DNA, to be easily distinguished and separated from plants not expressing the RNA, protein or polypeptide. Marker DNAs that can encode a protein that provides a distinguishable color to plant cells, such as the A1 gene encoding dihydroquercetin-4-reductase (Meyer et al., 1987) and the beta-glucoronidase gene (Jefferson et al., 1988), provides a specific morphological characteristic to the plant such as dwarf growth or a different shape of the leaves, or provides a selective advantage to cells which express the protein, e.g., resistance to chloramphenicol, methotrexate, hygromycin, streptomycin, spectinomycin, bleomycin, bromoxynil, sulfonamide, glyphosate or phosphinothricin. Other examples of marker DNAs are those that confer to plants: stress tolerance, such as is provided by the gene encoding superoxide dismutase as described in EPA 402,222, disease or pest resistance, such as is provided by a gene encoding a Bacillus thuringiensis endotoxin conferring insect resistance as described in EPA 300,291, or a gene encoding a bacterial peptide that confers a bacterial resistance as described in EPA 401,673.

In one embodiment, a marker DNA encodes a protein that inhibits or neutralizes the action of herbicides such as: the sfr gene and the sfrv gene, encoding enzymes conferring resistance to glutamine synthetase inhibitors such as Biolaphos and phosphinotricine as described in EPA 400,544; genes encoding modified target enzymes for certain herbicides that have a lower affinity for the herbicides than naturally produced endogenous enzymes, such as a modified glutamine synthetase as target for phosphinotricine as described in EPA 240,792 and a modified 5-enolpyruvylshikimate-3 phosphate synthase as a target for glyphosate as described in EPA 218,571. Other examples include a marker DNA encoding a protein which neutralizes the action of the herbicide bromoxynil (Stalker et al., 1988); the herbicide sulfonylurea (Lee et al., 1988), or the herbicide 2,4 D (presented at the 2nd International Symposium of Plant Molecular Biology, Jerusalem, Nov. 13-18, 1988).

Of course, many examples of suitable marker genes are known to the art and can be employed in the practice of the invention.

Included within the terms selectable or screenable marker or reporter genes are also genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA; and proteins that are inserted or trapped in the cell wall (e.g., proteins that include a leader sequence such as that found in the expression unit of extensin or tobacco PR-S).

With regard to selectable secretable markers, the use of a gene that encodes a protein that becomes sequestered in the cell wall, and which protein includes a unique epitope is considered to be advantageous. Such a secreted antigen marker would ideally employ an epitope sequence that would provide low background in plant tissue, a promoter-leader sequence that would impart efficient expression and targeting across the plasma membrane, and would produce protein that is bound in the cell wall and yet accessible to antibodies. A normally secreted wall protein modified to include a unique epitope would satisfy all such requirements.

One example of a protein suitable for modification in this manner is extensin, or hydroxyproline rich glycoprotein (HPRG). However, any one of a variety of extensins and/or glycine-rich wall proteins (Keller et al., 1989) could be modified by the addition of an antigenic site to create a screenable marker.

Possible selectable markers for use in connection with the present invention include, but are not limited to, a neo gene (Potrykus et al., 1985) which codes for kanamycin resistance and can be selected for using kanamycin, G418, and the like; a bar gene which codes for bialaphos resistance; a gene which encodes an altered EPSP synthase protein (Hinchee et al., 1988) thus conferring glyphosate resistance; a nitrilase gene such as bxn from Klebsiella ozaenae which confers resistance to bromoxynil (Stalker et al., 1988); a mutant acetolactate synthase gene (ALS) which confers resistance to imidazolinone, sulfonylurea or other ALS-inhibiting chemicals (EPA 154,204); a methotrexate-resistant DHFR gene (Thillet et al., 1988); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide (CTP).

Screenable markers that may be employed include, but are not limited to, a β-glucuronidase or uidA gene (GUS) which encodes an enzyme for which various chromogenic substrates are known; an R-locus gene, which encodes a product that regulates the production of anthocyanin pigments (red color) in plant tissues (Dellaporta et al., 1988); a beta-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., 1986), which allows for bioluminescence detection; or even an aequorin gene (Prasher et al., 1985), which may be employed in calcium-sensitive bioluminescence detection, or a green fluorescent protein gene (Niedz et al., 1995). The presence of the lux gene in transformed cells may be detected using, for example, X-ray film, scintillation counting, fluorescent spectrophotometry, low-light video cameras, photon-counting cameras, or multiwell luminometry. It is also envisioned that this system may be developed for populational screening for bioluminescence, such as on tissue culture plates, or even for whole plant screening.

The second promoter, which controls the marker DNA, can also be selected and isolated in a well-known manner so that the marker DNA is expressed either selectively in one or more specific tissues or specific cells, or constitutively in the entire plant, as desired depending on the nature of the RNA or protein encoded by the marker DNA. For example, if the marker DNA encodes herbicide resistance, it may be useful to have the marker DNA expressed in all cells of the plant, using a strong constitutive second promoter such as a CaMV 35S promoter (Odell et al., 1985; Hull and Howell, 1987), the promoter of the nopaline synthetase gene (“PNOS”) of the Ti-plasmid (Herrera-Estrella, 1983) or the promoter of the octopine synthase gene (De Greve et al., 1982). If the marker DNA encodes a protein conferring disease resistance, it may be useful to have the marker DNA selectively expressed in wounded tissue by using, for example, a TR promoter such as the TR1′ or TR2′ promoter of the Ti-plasmid (Velten et al., 1984). If the marker DNA encodes herbicide resistance, it may be useful to have the marker DNA selectively expressed in green tissue by using, for example, the promoter of the gene encoding the small subunit of Rubisco (EPA 400,544). If the marker DNA encodes a pigment, it may be useful to have the marker DNA expressed in specific cells, such as petal cells, leaf cells or seed cells, preferably in the outside layer of the seed coat.

If more than one marker DNA is present in the recombinant DNA molecule, all the marker DNAs can be under the control of a single promoter, but preferably, each marker DNA is under the control of its own separate promoter. More preferably, each marker DNA is under the control of its own promoter and encodes a different RNA or protein, providing different distinguishable characteristics to a transformed plant.

The recombinant DNA molecule can also comprise at least one marker DNA that is under the control of, and is fused at its 5′ end to, a promoter and is fused at its 3′ end to suitable transcription termination signals, including a polyadenylation signal.

4. Transit and Signal Peptides

When it is desired to have the gene product encoded by the cell ablation DNA or the gene product encoded by the marker DNA transported from the cytoplasm into chloroplasts or mitochondria of the cells of transformed plants, the recombinant DNA molecule can further include a DNA sequence encoding a transit peptide. The DNA sequence encoding the transit peptide is operably linked to the cell ablation DNA or to the marker DNA.

By “transit peptide” is meant a polypeptide fragment which is normally associated with a chloroplast or mitochondrial protein or subunit of the protein and is produced in a cell as a precursor protein encoded by the nuclear DNA of the cell. The transit peptide is responsible for the translocation process of the nuclear-encoded chloroplast or mitochondrial protein or subunit into the chloroplast or the mitochondria, and during such a process, the transit peptide is separated or proteolytically removed from the chloroplast or mitochondrial protein or subunit.

One or more of such additional DNA's can be provided in the recombinant DNA molecule of this invention for transporting one or more proteins as generally described in EPA 402,596, EPA 402,222 and in Van den Broeck et al. (1985); Schatz (1987); and Boutry et al. (1987). An example of a suitable transit peptide for transport into chloroplasts is the transit peptide of the small subunit of the enzyme RUBP carboxylase (EPA 402,596) and an example of a transit peptide for transport into mitochondria is the transit peptide of the enzyme Mn-superoxide dismutase.

When it is desired to have a gene product encoded by a cell ablation DNA or a gene product encoded by a marker DNA secreted out of the intercellular areas of plant cells in which they are expressed, or out of the tissue in which they are expressed, the recombinant DNA molecule can further include a DNA sequence encoding a secretory signal peptide. The DNA sequence encoding the secretory signal peptide is operably linked to the DNA encoding the cell ablating gene product or to the marker DNA.

By “secretory signal peptide” is meant a natural polypeptide fragment which is, particularly in eukaryotic cells, associated during translocation with proteins that are normally secreted from cells or an artificial polypeptide fragment which, when associated during translocation with a protein or polypeptide, provokes its secretion from cells.

B. Plant Transformation and Identification of Transformed Plants

1. Methods for Plant Transformation

A variety of techniques are available and known to those skilled in the art for introduction of vectors or expression cassettes comprising the recombinant DNA molecule of the invention into a plant cell host. Those skilled in the art will appreciate that the choice of method might depend on the type of plant, i.e., monocotyledonous or dicotyledonous, targeted for transformation. Some techniques may require a particular vector. For instance, for transformation using A. tumefaciens or A. rhizogenes, it is particularly preferred to use the binary type vectors of Ti and Ri plasmids of Agrobacterium spp. Ti-derived vectors transform a wide variety of higher plants, including monocotyledonous and dicotyledonous plants (see e.g., Pacciotti et al., 1985; Byrne et al., 1987; Sukhapinda et al., 1987; Lorz et al., 1985; Potrykus, 1985; Park et al., 1985; Hiei et al., 1994). The use of T-DNA to transform plant cells has received extensive study and is amply described (EP 120516; Hoekema, 1985, Knauf et al., 1983; and An. et al., 1985.

Thus, plant species may be transformed with the recombinant DNA molecule invention by any method including but not limited to the DNA-mediated transformation of plant cell protoplasts, microprojectile bombardment, DNA injection, electroporation, infection or co-cultivation with Agrobacterium (see EP 295,959; Fromm et al., 1986; Kline et al., 1987; U.S. Pat. No. 4,945,050; De Block et al., 1989; Everett et al., 1987; McCabe et al., 1988; Hinchee et al., 1988; Chee et al., 1989; Christou et al., 1989; EP 301749; Hiei et al., 1994; Gordon Kamm et al., 1990; Fromm et al., 1990; Crossway et al., 1986; Riggs et al., 1986; Paszkowski et al., 1984; Weissinger et al., 1988; Sanford et al., 1987; Christou et al., 1988; Datta et al., 1990; Klein et al., 1988; Svab et al., 1990; Koziel et al., 1993; Shimamoto et al., 1989; Christou et al., 1991; EP 0 332 581; Vasil et al., 1993; and Weeks et al., 1993) with subsequent regeneration of the plant from the transformed protoplasts or cells in accordance with procedures well known in the art.

Any plant tissue capable of subsequent clonal propagation, whether by organogenesis or embryogenesis, may be transformed with a recombinant DNA molecule of the invention. The term “organogenesis,” as used herein, means a process by which shoots and roots are developed sequentially from meristematic centers; the term “embryogenesis,” as used herein, means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristems, axillary buds, and root meristems), and induced meristem tissue (e.g., cotyledon meristem and hypocotyl meristem).

Transformation of plants can be undertaken with a single DNA molecule or multiple DNA molecules (i.e., co-transformation), and both these techniques are suitable for use with the expression cassettes of the present invention. Numerous transformation vectors are available for plant transformation, and the expression cassettes of this invention can be used in conjunction with any such vectors. The selection of vector will depend upon the preferred transformation technique and the target species for transformation.

Many vectors are available for transformation using Agrobacterium tumefaciens. These typically carry at least one T-DNA border sequence and include vectors such as pBIN19 (Bevan, 1984). In one preferred embodiment, the expression cassettes of the present invention may be inserted into either of the binary vectors pCIB200 and pCIB2001 for use with Agrobacterium. These vector cassettes for Agrobacterium-mediated transformation were constructed in the following manner. pTJS75kan was created by NarI digestion of pTJS75 (Schmidhauser & Helinski, 1985) allowing excision of the tetracycline-resistance gene, followed by insertion of an AccI fragment from pUC4K carrying an NPTII (Messing & Vierra, 1982; Bevan et al., 1983; McBride et al., 1990). XhoI linkers were ligated to the EcoRV fragment of pCIB7 which contains the left and right T-DNA borders, a plant selectable nos/nptII chimeric gene and the pUC polylinker (Rothstein et al., 1987), and the XhoI- digested fragment was cloned into SalI-digested pTJS75kan to create pCIB200 (see also EP 0 332 104). pCIB200 contains the following unique polylinker restriction sites: EcoRI, SstI, KpnI, BglII, XbaI, and SalI. The plasmid pCIB2001 is a derivative of pCIB200 which was created by the insertion into the polylinker of additional restriction sites. Unique restriction sites in the polylinker of pCIB2001 are EcoRI, SstI, KpnI, BglII, XbaI, SalI, MluI, BclI, AvrII, ApaI, HpaI, and StuI. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

An additional vector useful for Agrobacterium-mediated transformation is the binary vector pCIB 10, which contains a gene encoding kanamycin resistance for selection in plants, T-DNA right and left border sequences and incorporates sequences from the wide host- range plasmid pRK252 allowing it to replicate in both E. coli and Agrobacterium. Its construction is described by Rothstein et al. (1987). Various derivatives of pCIB10 have been constructed which incorporate the gene for hygromycin B phosphotransferase described by Gritz et al. (1983). These derivatives enable selection of transgenic plant cells on hygromycin only (pCIB743), or hygromycin and kanamycin (pCIB715, pCIB717).

Methods using either a form of direct gene transfer or Agrobacterium-mediated transfer usually, but not necessarily, are undertaken with a selectable marker which may provide resistance to an antibiotic (e.g., kanamycin, hygromycin or methotrexate) or a herbicide (e.g., phosphinothricin). The choice of selectable marker for plant transformation is not, however, critical to the invention.

For certain plant species, different antibiotic or herbicide selection markers may be preferred. Selection markers used routinely in transformation include the nptII gene which confers resistance to kanamycin and related antibiotics (Messing & Vierra, 1982; Bevan et al., 1983), the bar gene which confers resistance to the herbicide phosphinothricin (White et al., 1990; Spencer et al., 1990), the hph gene which confers resistance to the antibiotic hygromycin (Blochinger & Diggelmann), and the dhfr gene, which confers resistance to methotrexate (Bourouis et al., 1983).

One such vector useful for direct gene transfer techniques in combination with selection by the herbicide Basta (or phosphinothricin) is pCIB3064. This vector is based on the plasmid pCIB246, which comprises the CaMV 35S promoter in operational fusion to the E. coli GUS gene and the CaMV 35S transcriptional terminator and is described in WO 93/07278. One gene useful for conferring resistance to phosphinothricin is the bar gene from Streptomyces viridochromogenes (Thompson et al., 1987). This vector is suitable for the cloning of plant expression cassettes containing their own regulatory signals. An additional transformation vector is pSOG35 which utilizes the E. coli gene dihydrofolate reductase (DHFR) as a selectable marker conferring resistance to methotrexate. PCR was used to amplify the 35S promoter (about 800 bp), intron 6 from the maize Adh1 gene (about 550 bp) and 18 bp of the GUS untranslated leader sequence from pSOG10. A 250 bp fragment encoding the E. coli dihydrofolate reductase type II gene was also amplified by PCR and these two PCR fragments were assembled with a SacI-PstI fragment from pBI221 (Clontech) which comprised the pUC19 vector backbone and the nopaline synthase terminator. Assembly of these fragments generated pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus check (MCMV) generated the vector pSOG35. pSOG19 and pSOG35 carry the pUC-derived gene for ampicillin resistance and have HindIII, SphI, PstI and EcoRI sites available for the cloning of foreign sequences.

Plants of the present invention may take a variety of forms. The plants may be chimeras of transformed cells and non-transformed cells; the plants may be clonal transformants (e.g., all cells transformed to contain the expression cassette); the plants may comprise grafts of transformed and untransformed tissues (e.g., a transformed root stock grafted to an untransformed scion in citrus species). The transformed plants may be propagated by a variety of means, such as by clonal or vegetative propagation. A dominant selectable marker (such as npt II) can be associated with the expression cassette to assist in breeding.

2. Production and Characterization of Stably Transformed Plants

Transgenic plant cells are placed in an appropriate selective medium for selection of transgenic cells which are then grown to callus. Shoots are grown from callus and plantlets generated from the shoot by growing in rooting medium. For some woody plant, dark and/or low light treatment improves adventitious shoot production. The various expression cassettes normally will be joined to a marker for selection in plant cells. Conveniently, the marker may be resistance to a biocide (particularly an antibiotic, such as kanamycin, G418, bleomycin, hygromycin, chloramphenicol, herbicide, or the like). The particular marker used will allow for selection of transformed cells as compared to cells lacking the DNA which has been introduced. Components of expression cassettes including transcription cassettes of this invention may be prepared from sequences which are native (endogenous) or foreign (exogenous) to the host. By “foreign” it is meant that the sequence is not found in the wild-type host into which the expression cassette is introduced. Chimeric expression cassettes will contain at least one region which is not native to the gene from which the transcription-initiation-region is derived.

To confirm the presence of the transgenes in transgenic cells and plants, a Southern blot analysis can be performed using methods known to those skilled in the art. Transgenes can be detected and quantitated by Southern blot, since they can be readily distinguished from nontransgene sequences by the use of appropriate restriction enzymes or by PCR. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts may be harvested, and/or the seed collected. The seed may serve as a source for growing additional plants with tissues or parts having the desired characteristics.

The invention will be further described by the following non-limiting examples.

### EXAMPLE I

The following are exemplary transformation protocols and media for plants, e.g., tomato, petunia and woody plants.

### Materials

I. General Media

Davis Germination Media (DGM)
20 g Sucrose
10 mls Nitsch Vitamins (100× Stock)
1×MS salts
pH 5.8 and bring to 1 liter
1% Difco Bacto or Sigma A agar (0.6% Difco Bacto Bitek agar)
Autoclave

Calgene Preculture Medium (CP)
30 g Sucrose
0.1 g Myo-inositol
0.2 g KH₂PO₄
1.5 mls Thiamine HCl (0.9 mg/ml Stock
1×MS salts
50 μl 2.0 mg Kinetin/ml Stock
10 μl 2.0 mg 2,4-D/ml
20 pH 6.0
Bring to 1 liter with
1% Bacto or Sigma A agar (0.6% Difco Bacto Bitek agar)
Autoclave

Davis Regeneration Medium (DRM)
30 g Sucrose
10 ml Nitsch Vitamins (100× Stock)
1×MS Stock
Bring to 1 liter
pH 5.8
1% Bacto or Sigma A agar for solid media (0.6% Difco Bacto Bitek agar)
Autoclave
Cool to 50° C. and add selective agents
2 ml 1 mg Zeatin/ml Stock, filter sterilize (to solubulize Zeatin use minimal amount of 1N HCl)
1 ml 500 mg Carbenicillin/ml, filter sterilize Stock (500 μg/ml final)
1 ml 100 mg Kanamycin/m, filter sterilize Stock (100 μg/ml final)
(Vancomycin at 100 mg/ml final concentration can be used if Agrobacterium overrun is a problem)
Note: a powdered Murashige and Skoog basal medium with Gamborgs vitamins (Sigma, M 0404) that may be used.

TXD Medium
2 ml B5 Vitamins (500× Stock)
30 g Sucrose
2 ml 2 mg p-chlorophenoxy acetic acid/ml Stock
10 μl 0.5 mg Kinetin/ml Stock
1×MS Stock
Bring to 1 liter

½ MSO
2 ml B5 Vitamins (500× Stock)
30 g Sucrose
½×MS salts
pH to 5.8
Bring to 1 liter
1% Bacto or Sigma A agar (0.6% Difco Bacto Bitek agar)
Autoclave

MS Salts

| Stock 1 | Stock 2 | Stock 3 | Stock 4 |
|---|---|---|---|
| 82.5 g NH₄NO₃ | 17.0 g KH₂PO₄ | 18.0 g MgSO₄ | 11.2 g Sequestrene |
| 95.0 g KNO₃ | 0.62 g H₃BO₃ | 1.69 g MnSO₄H₂O | (Fe33O) |
| 16.67 g GaCl₂ | 0.0025 g CoCl₂-6H₂O | 1.06 g ZnSO₄-7H₂O | per liter |
| per liter | 0.025 g NaMoO₄-2H₂O | 0.0025 g CuSO₄-5H₂O | |
| | per liter | 0.083 g KI | |
| | | per liter | |

For one liter use:
20 ml 10 ml 10 ml 5 ml

100× Nitsch Vitamins
100 mg Myo-inositol
5 mg Nicotinic Acid
0.5 mg Pyridoxine HCl
0.5 mg Thiamine HCl
0.5 mg Folic Acid
0.5 mg Biotin
2 mg Glycine
per liter

500× B5 Vitamins
50 g Myo-inositol
5 mg Thiamine HCl
0.5 mg Nicotinic Acid
0.5 g Thiamine HCl
0.5 g Pyridoxine
per liter
   II. Media for Woody Plants (Plants to be used in a transformation experiment are kept under high light intensity, e.g., 50 μEinsteins or more)

Maintenance Medium (1 L)

(For culture of plant prior to putting on leaf expansion medium.)
4.3 g Murashige and Skoog medium (MS) salts
30 g sucrose
7.5 g agar
100 mg myo-inositol

Vitamins:
0.8 mg/l Thiamine
1.0 Pyridoxine
1.0 Nicotinic acid
4.0 Glycine
0.08 Biotin

PGR:
0.1 mg Naphthalene acetic acid (NAA)
1.0 mg Benzyl adenine (BA)
3.0 mg Kinetin
pH 5.6

Leaf Expansion Medium.

About 4-5 weeks before transformation, transfer single shoots horizontally into separate containers. Same as Maintenance medium except use these PGRs:
0.1 mg/l NAA
8.3 mg/l isopentennyladenine (2 ip)

Co-Culture Medium (1 L)
4.3 g MS salts
LS vitamins
   100 mg/l Myo-inositol
   0.4 mg/l Thiamine HCl
30 g sucrose
0.3 mg/l Indole Butyric Acid
2.0 mg/l Thidiazuron (TDZ)
2.5 g gelrite

After autoclaving, add, by filter sterilization
1 ml acetosyringone stock (see above)
1 ml betaine phosphate stock (see above)

LB Medium for Growing Agrobacterium (1 L)
10 g/l Bacto-tryptone
5 Bacto-yeast extract
5 NaCl
18 Bacto-agar
after autoclaving add antibiotics for appropriate selection

Agrobacterium Virulence Induction Medium (1 L)
5.88 g/l Citric Acid (trisodium dihydrate)
20 g/l sucrose
after autoclaving, add: Acetosyringone stock to achieve 100 μM and betaine phosphate to achieve 1 mM

MSO+ (1 L)
2.15 g MS salts
500 mg cefotaxime, filter sterilized into sterile medium.

Selection Medium (1 L)
4.3 g MS
LS vitamins (see above)
30 g sucrose
2 mg TDZ
0.3 mg IBA

after autoclaving, filter in
350 mg cefotaxime
100 mg kanamycin

### Methods

Germination of Seeds

Weigh 1.5 g of seed (approximately 700 seeds, may vary with cultivar). Sterilize in a 200 ml solution of 20% bleach and 2% Liqui-Nox for 20 minutes. Pour off the bleach solution (a small nylon mesh that has been sterilized is helpful to catch the seeds). Rinse quickly with sterile distilled water. Then rinse for 5 minutes in 200 ml of sterile water, mixing every minute. Repeat the rinse three times. Place seeds on sterile dry paper towels to blot excess moisture. Place approximately 70 seeds in a vessel (e.g., the size of a baby food jar) with 25 ml of Davis Germination Media (DGM). Seeds will also germinate on Murashige and Skoog medium with 5 g/l sucrose. Germinate at 25° C. on a light rack, 16 hour light period, for 16-22 days.

Preculture

Trim the ends of cotyledons with a sterile scalpel, or snip with sterile scissors. Then place the cut cotyledons (abaxial down) on plates (30-35 cotyledons/plate) containing approximately 15 ml of Calgene Preculture Medium (CP). Keep cotyledons on these plates at 25° C., continuous light (45-50 microeinsteins m⁻² s⁻¹) for 1 to 2 days or with a 16 hour light photoperiod. These growth conditions are continued until rooted shoots are transferred to soil. Cotyledons should be in good contact with the medium during all culture steps.

Inoculation/Coculture

Remove cotyledons from CP plates and place cotyledons from 3 plates into 100 ml of 5×10⁸ Agrobacterium +200 μM Acetosyringone. Leave cotyledons in the Agrobacterium for 5 and up to 45-60 minutes. Blot cotyledons dry on sterile paper towels and replace them on the same CP plates for 2 days. Agrobacterium is usually from an overnight culture that has been transferred 4-6 hours prior to inoculation into antibiotic-free medium, and diluted in TXD medium. Grow Agrobacterium at 30° C. in LB plus selection (5×10⁸ cells=0.17 OD₆₆₀).

Selection/Regeneration

Remove the cotyledons from the CP plates and place onto Davis Regeneration Medium (DRM) with approximately 20 cotyledons/plate. When shoots are produced, place the cotyledons in larger petri plates of DRM. Trim as much callus from the shoots as possible, without damaging the shoots with each transfer. When shoots have elongated to 1+ cm they may be cut away from other material and placed on ½ MSO in 100×25 mm petri plates (remove all callus before rooting). When roots are formed, the rooted shoots are removed and washed in water. Optionally, rinse shoots in a 10% bleach solution for 2 minutes, then rinse in water for 1 minute, dip in Captan (1 g/L), and rinse in water. The rooted shoots are then placed in sterile plastic Magenta boxes with sterile ProMix and are kept covered for 1-2 weeks. Standard growth chamber conditions are employed for plants. After 1-2 weeks the tops of the boxes are opened slightly allowing the plant to acclimate. Every few days open lids more to allow the plants to “harden off” slowly, keep soil moist with 100 ppm Peter's 20-20-20. Lids are removed and regenerated plants transplanted in 2-6 weeks.

For woody plants, inoculate LB plates with Agrobacterium and incubate 2-4 days at 28° C. Harvest Agrobacterium with Virulence Induction Medium. Harvest leaves, wound each leaf, and immerse in Agrobacterium for about 5 minutes. Remove leaves from slurry and place on dry sterile filter paper for blotting. Leave them to dry for 10 minutes or more, then place them right side up, on co-culture medium. Seal plates, wrap in aluminum foil, and place plates upside down (to prevent condensation) in a sealed plastic bag. Leave for 3 days at 25° C.

### EXAMPLE II

Four expression cassettes were prepared. Two of the expression cassettes included a pistil-specific promoter, the SP41 promoter (Ori et al., 1990; Sessa et al., 1995), linked to either a marker DNA, e.g., a DNA encoding beta-glucuronidase (GUS), or a cell ablation DNA, e.g., an open reading frame for barnase. The other two expression cassettes included a stamen-specific promoter, the 108GRP promoter, linked to either a marker DNA, e.g., a beta-glucuronidase gene, or a cell ablation DNA, e.g., an open reading frame for barnase.

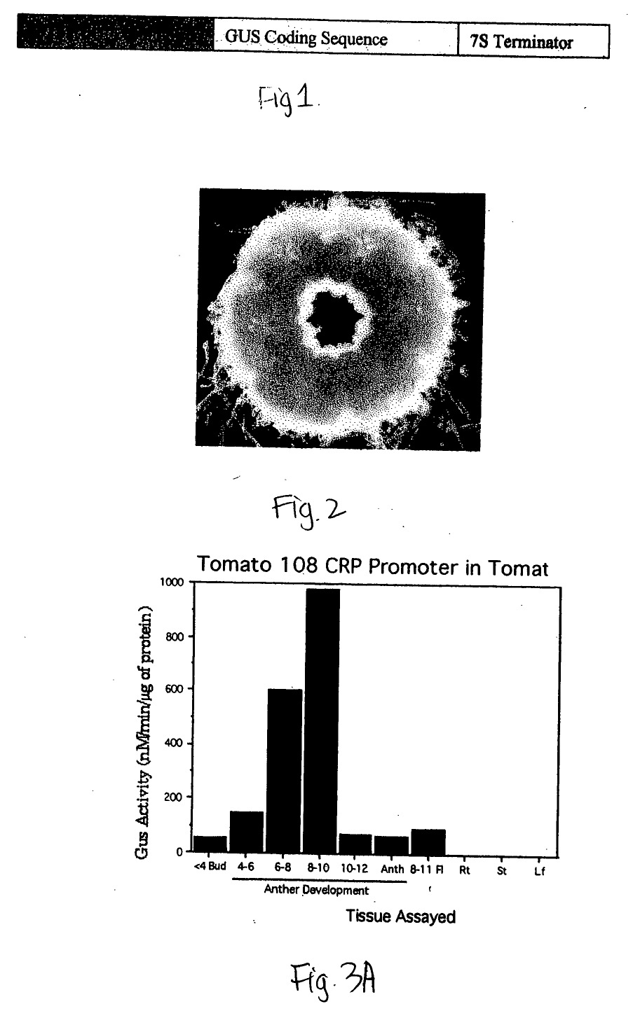

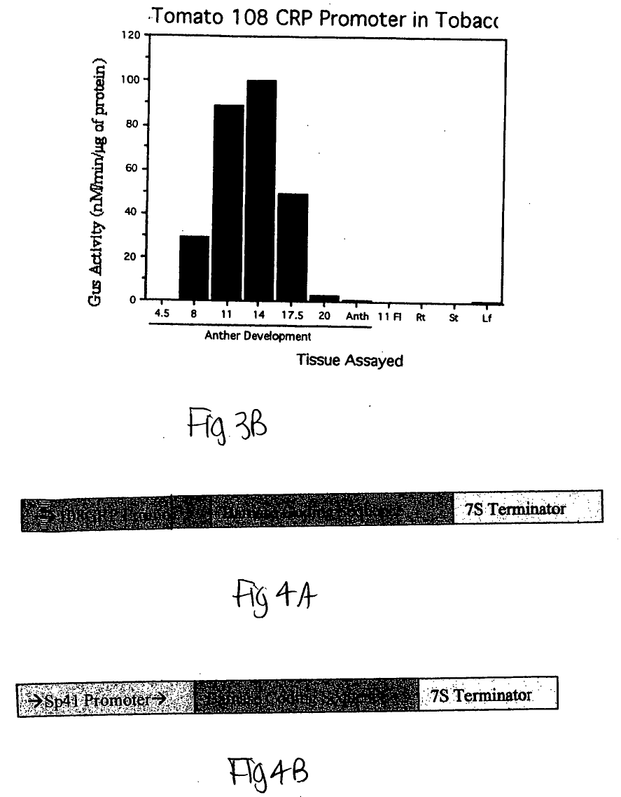

To determine the tissue specificity of the 108GRP promoter, a 108GRP-GUS expression cassette (FIG. 1) was introduced to tomato cells and plants regenerated therefrom. As shown in FIG. 2, GUS activity was localized in the anther of the tomato flower and was not in other portions of the flower. Staining of other organs of the tomato plant showed only very weak levels of GUS activity. Moreover, as shown in FIG. 3, GUS expression was highest in flowers 6-10 mm in length for tomato and in flowers 11-17 mm in length for tobacco.

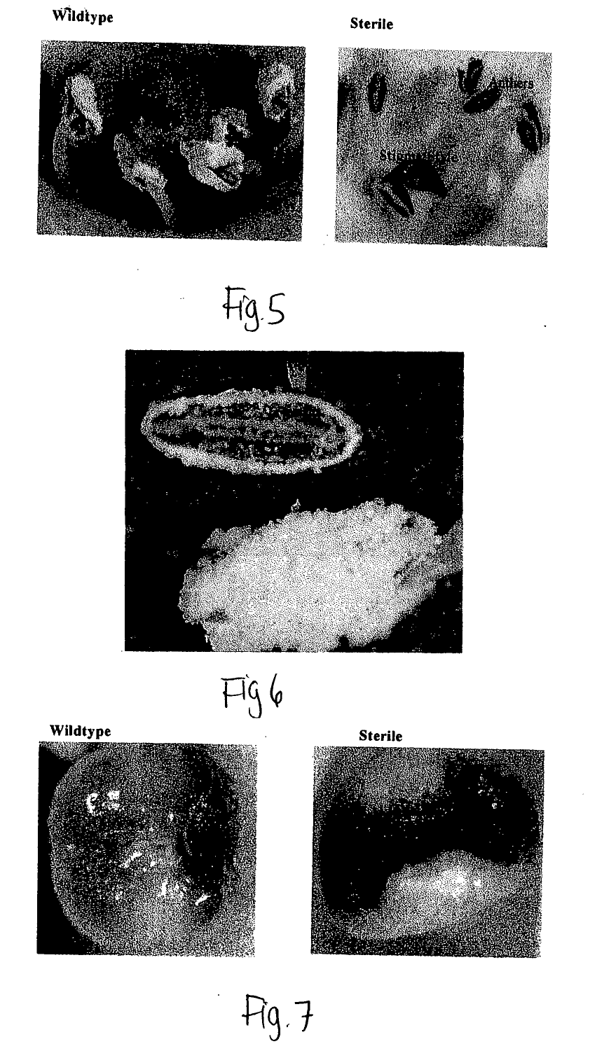

Tobacco plants having the 108GRP-barnase expression cassette (FIG. 4A) and the SP41-barnase expression cassette (FIG. 4B) were prepared. As shown in FIGS. 5 and 6, anthers from a wild-type plant had a large amount of viable pollen on the surface of the anther, whereas the anthers of the sterile plant had no pollen. Moreover, surprisingly, the sterile plants had more flowers and remained in flower longer than the corresponding nontransgenic plant.

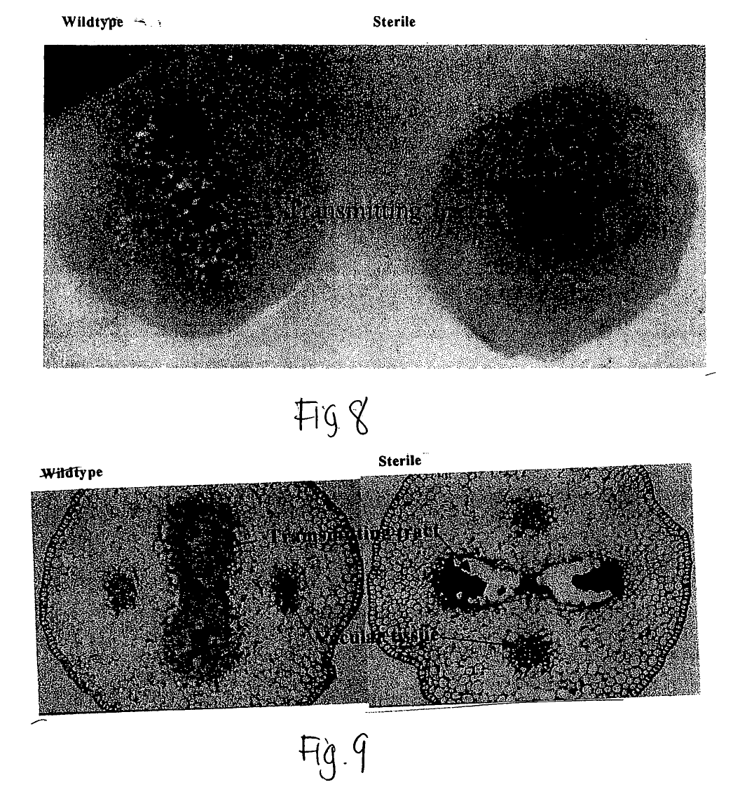

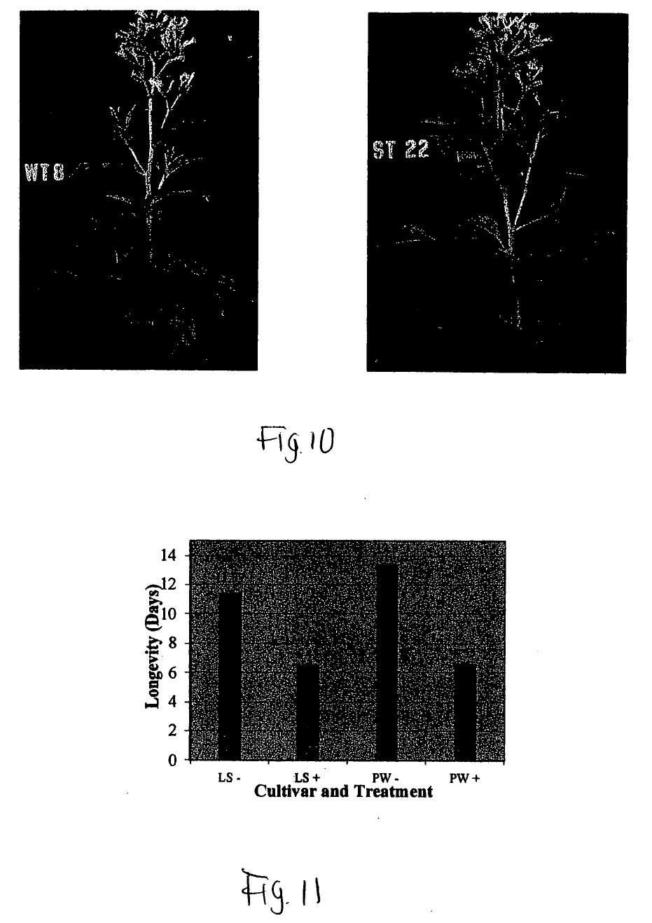

The SP41-barnase expression cassette clearly had a striking effect on the development of the stigma and, consequently, its function and ability to be fertilized. The necrosis of the stigma due to barnase expression continued from the stigma through the style (FIG. 7) but had no phenotype in the ovary. The tissue affected corresponds to the transmitting tract, which is where the SP41 promoter has been shown to have its highest level of expression.

Pistils from greenhouse grown tobacco plants were cut midway up the style with a razor blade, mounted and photographed with a stereomicroscope. FIGS. 8-9 demonstrate the ablation of the transmitting tract within the style of tobacco plants carrying the SP41-barnase expression cassette. The lack of the transmitting tract limits the female fertility of the plant and therefore inhibits seed set and fruit development in such a plant, even in the presence of viable pollen.

FIG. 11 shows results obtained from petunia transformed with the 108GRP-barnase expression cassette. The data in FIG. 11 clearly show that the presence of a male sterility gene in petunia can approximately double the life of flowers.

### REFERENCES

Albani et al., Plant J., 2:331 (1992).
Altenbach et al., Plant Molecular Biol., 8:239 (1987).
An et al., EMBO J. 4:277 (1985).
Ballas et al., Nucleic Acids Res. 17:7891 (1989).
Bechtold et al., CR Acad. Sci. Paris Life Sci., 316:1194 (1993).
Bevan et al., Nature, 304:184 (1983).
Bevan, Nucl. Acids Res. (1984).
Blochinger & Diggelmann, Mol Cell Biol, 4:2929.
Bourouis et al., EMBO J., 2:1099 (1983).
Boutry et al., Nature, 328:340 (1987).
Bowman et al., Devel., 114:599 (1992).
Braun et al., Plant Cell, 2:153 (1990).
Byrne et al. Plant Cell Tissue and Organ Culture, 8:3 (1987).
Callis et al., Genes Dev., 1:1183 (1987).
Chee et al. Plant Physiol., 91:1212 (1989).
Christou et al. Proc. Natl. Acad. Sci. USA, 86:7500 (1989).
Christou et al., Biotechnology, 9:957 (1991).
Christou et al., Plant Physiol., 87:671 (1988).
Chuong and Beversdorf, Plant Sci., 39:219 (1985).
Coen et al., Cell, 63:1311 (1991).
Crossway et al., BioTechniques, 4:320 (1986).
Dale et al., Plant Breeding, 111:1 (1993).
Datta et al., Bio/Technology, 8:736 (1990).
Dayhoff et al., Atlas of Protein Sequence and Structure (Natl. Biomed. Res. Found., Washington, D.C. (1978)).
De Blaere et al., Meth. Enzymol. 143:277 (1987).
De Block et al. Plant Physiol. 91:694 (1989).
De Greve et al., J. Mol. Appl. Genet., 1:499 (1982).
Dellaporta et al., in Chromosome Structure and Function, pp. 263-282 (1988).
Denecke et al., The Plant Cell, 2:51 (1990).
E. B. Swanson, M. P. Coumans, S. C. Wu, T. L. Barby and W. D. Beversdorf, Plant Cell Reports 6:94 (1987).
Everett et al. Bio/Technology, 5:1201 (1987).
Fishlock, D., The PROSAMO Report, published by the Laboratory of the Government Chemist, Queens Road, Teddington, Middlesex, UK TW11 0LY.
Friedrich et al., Plant Mol. Biol., 29:959 (1995).
Fromm et al., Nature, 319:791 (1986).
Fromm et al., Bio/Technology, 8:833 (1990).
Gasser et al., The Plant Cell, 1:15 (1989).
Gielen et al., EMBO J., 3:835 (1984).
Goldberg et al., Plant Cell, 5:1217 (1993).
Goldman et al., EMBO J., 13:2976 (1994).
Gordon-Kamm et al., Plant Cell, 2:603 (1990).
Grimsley et al., EMBO Journal, 5:641 (1986).
Gritz et al., Gene, 25:179 (1983).
Gruber, et al., In Methods in Plant Molecular Biology & Biotechnology” in Glich et al., (Eds. pp. 89-119, CRC Press, 1993).
Guerineau et al., Mol. Gen. Genet., 262:141 (1991).
Hamilten et al., Sexual Plant Reproduction, 2:208 (1989).
Haseloff and Gerlach, Nature, 334:585 (1988).
Hemould et al., Proc. Natl. Acad. Sci. USA, 90:2370 (1993).
Herrera-Estrella, Nature, 303:209 (1983).
Hiei et al., Plant J., 6:271 (1994).
Hinchee et al., Biotechnology, 6:915 (1988).
Hoekema, In: The Binary Plant Vector System. Chapter V. Offset-drukkerij Kanters B. V.; Alblasserdam (1985).
Horiuchi et al., J. Biochem, 103:408 (1988).
Horsch et al., Science, 227:1229 (1985).
Hull and Howell, Virology, 86:482 (1987).
Ikuta et al., Biotech., 8, 241 (1990).
Ingelbrecht et al., Plant Cell, 1:671 (1989).
Jefferson et al., Proc. Natl. Acad. Sci. USA, 83:8447 (1988).
Jeon et al., Plant Mol. Biol., 39:35 (1999).
Jofuku and Goldberg, The Plant Cell, 1:1079 (1989).
Joshi et al., Nucleic Acid Res., 15:9627 (1987).
Kapoor et al., Plant Cell, 14:2353 (2002).
Katz et al., J. Gen. Microbiol., 129, 2703 (1983).
Kawata et al., Eur. J. Biochem, 176:683 (1988).
Keller et al., EMBO J., 8, 1309 (1989).
Klein et al., Nature, 327:70 (1987).
Klein et al., Bio/Technology, 6:559 (1988).
Klein et al., Plant Physiol., 91:440 (1988).
Klein et al., Proc. Natl. Acad. Sci. USA, 85:4305 (1988).
Knauf et al., Genetic Analysis of Host Range Expression by Agrobacterium In: Molecular Genetics of the Bacteria-Plant Interaction, Puhler, A. ed., Springer-Verlag, New York, p. 245 (1983).
Koziel et al., Biotechnology, 11:194 (1993).
Krebbers et al., Plant Physiol., 87:859 (1988).
Kunkel et al., Methods in Enzymol., 154:367 (1987).
Kunkel, Proc. Natl. Acad. Sci. USA, 82:488 (1985).
Lang et al., Genetics Soc. Am., 137:1101 (1991).
Larsson et al., Biochim. Biophys. Acta, 1577:53 (2002).
Lecoq et al., Molec. Plant Microbe Interact., 6:403 (1993).
Lee et al., EMBO J., 7:1241 (1988).
Lexis et al., J. Virol., 64:1726 (1990).
Lindsey et al., Transgenic Research, 2:3347 (1993).
Lorz et al. Mol. Gen. Genet., 199:178 (1985).
Maeser et al., Mol. Gen. Genet., 230:170 (1991).
Maki et al., In Methods in Plant Molecular Biology & Biotechnology, Glich et al. (Eds. pp. 67-88 CRC Press, 1993).
Mandel et al., Cell, 71:133 (1992).
Mandel et al., Nature, 377:522 (1995).
Margraff et al., Experimentia, 36:846 (1980).
Mariani et al., Nature, 347:737 (1996).
Mariani et al., Nature, 347:737 (1990).
Mariani et al., Nature, 357:384 (1992).
Mascarenhas, Bot. Rev., 41:259 (1975).
McBride et al., Plant Molecular Biology, 14:266 (1990).
McCabe et al., Biotechnology, 6:923 (1988).
McClure et al., Nature, 342:955 (1989).
McElroy et al., Mol. Gen. Genet., 231:150 (1991).
Meer et al., Plant Mo. Biol., 15:95 (1990).
Messing & Vierra, Gene, 19:259 (1982).
Meulewater et al., Virology, 177:1 (1990).
Meyer et al., Nature, 330:677 (1987).
Moffatt et al., Plant Physiol., 86:1150 (1980).
Mogen et al., Plant Cell, 2:1261 (1990).
Munroe et al., Gene, 91:151 (1990).
Niedz et al., Plant Cell Reports, 14, 403 (1995).
Odell et al., Nature, 313:810 (1985).
Okamuro et al., Biochemistry of Plants, 15:1 (1989).
Ori et al., EMBO J., 9:3429 (1990).
Ow et al., Science, 234, 856 (1986).
Owens et al., Weed Science, 21:63 (1973).
Pacciotti et al. Bio/Technology, 3:241 (1985).
Park et al., J. Plant Biol., 38:365 (1985).
Paszkowski et al., EMBO J., 3:2717 (1984).
Phillips et al., In Corn & Corn Improvement, 3rd Edition 10 Sprague et al. (Eds.) pp. 345-387, American Society of Agronomy Inc. et al. 1988.
Plant Tissue and Cell Culture, Plant Biology 3, A. R. Liss, Inc. N.Y. (1987).
Plasternak et al., Proc. Natl. Acad. Sci. USA, 80:5355 (1983).
Potrykus Mol. Gen. Genet., 199:183 (1985).
Prasher et al., Biochem. Biophys. Res. Comm., 126, 1259 (1985).
Proceedings of the 3rd International Symposium on The BioSafety Results of Field Tests of Genetically Modified Plants and Microorganisms, Monterey, Calif., D. D. Jones (1994).
Proudfoot, Cell, 64:671 (1991).
Raybould & Grey, J. Applied Ecology, 30:199 (1993).
Riggs et al., Proc. Natl. Acad. Sci. USA, 83:5602 (1986).
Rothstein et al., Gene, 53:153 (1987).
Sambrook et al., Molecular Cloning. A Laboratory Manual. Ed. Cold Spring Harbor Laboratory (1989).
Sanfacon et al., Genes Dev., 5:141 (1991).
Sanford et al., Particulate Science and Technology, 5:27 (1987).
Schatz. Eur. J. of Bioch., 165:1 (1987).
Schmidhauser & Helinski, J. Bacteriol., 164:446 (1985).
Scolfield and Crouch, J. Biol. Chem., 262:12202 (1987).
Second International Symposium of Plant Molecular Biology, Jerusalem, Nov. 13-18, 1988.
Sessa et al., Plant Mol. Biol., 29:969 (1995).
Shimamoto et al., Nature, 338:274 (1989).
Smith et al., Mol. Gen. Genet., 222:9 (1990).
Spena et al., Mol. Gen. Genet., 227:205 (1991).
Spena et al., Theor. Appl. Genet., 84:520 (1992).
Spencer et al., Theor. Appl. Genet, 79:625 (1990).
Stalker et al., In: Genetic Improvements of Agriculturally Important Crops, Ed: R. T. Fraley, N. M. Frey and J. Schell, Cold Spring Harbor Laboratories (1988).
Stalker et al., Science, 242, 419 (1988).
Sukhapinda et al., Plant Mol. Biol., 8:209 (1987).
Sutcliffe, PNAS USA, 75, 3737 (1978).
Svab et al., Proc. Natl. Acad. Sci. USA, 87:8526 (1990).
Tepfer, Biotechnology, 11:1125 (1993).
The bio-safety results of field tests of genetically modified plants and microorganisms. Proceedings of the 2nd International Symposium on The Biosafety Results of Field Tests of Genetically Modified Plants and Microorganisms, Goslar, Germany, 1992.
The field release of transgenic plants. The British Crop Protection Council. Brighton Crop Protection Conference: Pests and Diseases, Vols. I, II and III.
Thillet et al., J. Biol. Chem., 263, 12500 (1988).
Thompson et al., EMBO J., 6:2519 (1987).
Thorsness et al., Plant Cell, 5:253 (1993).
Tomes et al., In: Gamborg and Phillips (Eds.) Plant Cell, Tissue and Organ Culture: Fundamental Methods, Springer Verlag, Berlin (1995).
Trobner et al., EMBO J., 11:4693 (1992).
Tsuchiya et al., Plant Cell Physiol., 36:487 (1995).
Tunen et al., Plant Mol. Biol., 12:536 (1989).
Turner et al., Molecular Biotechnology, 3:225 (1995).
Twell et al., Genes Dev., 5:496 (1991).
University of California, Risk assessment in agricultural biology: proceedings of an international conference, 1990, Casper, R., & Landsman, J., 1992.
Van den Broeck et al., Nature, 313:358 (1985).
Van der Meer et al., Plant Cell, 4:253 (1992).
Vasil et al., Biotechnology, 11:1553 (1993).
Vasil et al., Mol. Microbiol., 3:371 (1989).
Velten and Schell, Nucleic Acids Research, 13:6981 (1985).
Velten et al., EMBO J., 3:2723 (1984).
Verheij et al., Rev. Biochem. Pharmacol., 91:92 (1981).
Von Heijne, NAR, 14:4683 (1986).
Walker and Gaastra, eds., Techniques in Molecular Biology (MacMillan Publishing Company, New York) (1983).
Weeks et al., Plant Physiol., 102:1077 (1993).
Weigel et al., Cell, 69:843 (1992).
Weissinger et al., Annual Rev. Genet., 22:421 (1988).
White et al., Nucl Acids Res, 18:1062 (1990).
Xu et al., Mol. Gen. Genetics, 239:58 (1993).
Yanofsky et al., Nature, 346:35 (1990).
Worrall et al., Plant Cell, 4:759 (1992).
Zukowsky et al., PNAS USA, 80, 1101 (1983).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to prepare a male-sterile plant having an increased number of flowers or flowers with increased longevity, comprising:
a) contacting cells of an ornamental plant with a recombinant DNA molecule comprising an expression cassette comprising a stamen-specific promoter operably linked to DNA segment encoding a gene product, the expression of which in a cell results in ablation of that cell, so as to yield transformed plant cells; and
b) regenerating the transformed plant cells to provide a differentiated transformed male-sterile plant having an increase in the number of flowers or flower longevity, or both, relative to the number of flowers or flower longevity in a corresponding plant which does not comprise the expression cassette.

2. The method of claim 1 wherein the promoter is a 108 promoter.

3. The method of claim 1 wherein the promoter is an A3 promoter, an A6 promoter or an A9 promoter.

4. The method of claim 1 wherein the promoter is transcribed in a developmental pattern or at a level that is substantially similar to that of a 108 promoter.

5. The method of claim 1 wherein the promoter is pollen-specific.

6. The method of claim 1 wherein the promoter is tapetal-specific.

7. The method of claim 1 wherein the DNA segment encodes a toxin, lipase, nuclease or protease.

8. The method of claim 1 wherein the DNA segment encodes a RNase.

9. The method of claim 1 wherein the DNA segment encodes barnase.

10. The method of claim 1 wherein the ornamental plant cells are cells of an annual plant.

11. The method of claim 10 which are Petunia or Clematis cells.

12. The method of claim 1 wherein the ornamental plant cells are cells of a perennial plant.

13. The method of claim 1 wherein the ornamental plant cells are cells of a woody plant.

14. The method of claim 13 wherein the ornamental plant cells are cells of a poplar, apple, or chokeberry.

15. The method of claim 1 wherein the ornamental plant cells are cells of a herbaceous plant.

16. The method of claim 1 wherein the expression cassette further comprises a transcription termination sequence.

17. The method of claim 16 wherein the transcription termination sequence is a 7S terminator sequence.

18. The method of claim 1 wherein flower longevity is increased at least 1.1-fold in the differentiated transformed male-sterile plant relative to the corresponding plant which lacks the expression cassette.

19. The method of claim 1 wherein flower longevity is increased at least 1.2-fold in the differentiated transformed male-sterile plant relative to the corresponding plant which lacks the expression cassette.

20. The method of claim 1 wherein flower longevity is increased at least 1.5-fold in the differentiated transformed male-sterile plant relative to the corresponding plant which lacks the expression cassette.

21. Progeny of the differentiated transformed male-sterile plant prepared by the method of claim 1.

22. A differentiated transformed male-sterile plant prepared by the method of claim 1.

23. A method to prepare a female-sterile plant having an increased number of flowers or flowers with increased longevity, comprising:
a) contacting cells of an ornamental plant with a recombinant DNA molecule comprising an expression cassette comprising a pistil-specific promoter operably linked to DNA segment encoding a gene product, the expression of which in a cell results in ablation of that cell, so as to yield transformed plant cells; and
b) regenerating the transformed plant cells to provide a differentiated transformed female-sterile plant having an increase in the number of flowers or flower longevity, or both, relative to the number of flowers or flower longevity of a corresponding plant which does not comprise the expression cassette.

24. The method of claim 23 wherein the promoter is the promoter for beta 1,3 glucanase, PSTMG07, PSTMG08, PSTMG4B12 or PSTMG3C9.

25. The method of claim 23 wherein the promoter is a SP41 promoter.

26. The method of claim 23 wherein the promoter is transcribed in a developmental pattern or at a level that is substantially similar to that of a SP41 promoter.

27. The method of claim 23 wherein the promoter is stigma- or style-specific.

28. The method of claim 23 wherein the promoter is ovary- or ovule-specific.

29. The method of claim 23 wherein the DNA segment encodes a toxin, lipase, nuclease or protease.

30. The method of claim 29 wherein the DNA segment encodes a RNase.

31. The method of claim 23 wherein the DNA segment encodes barnase.

32. The method of claim 23 wherein the ornamental plant cells are cells of an annual plant.

33. The method of claim 23 wherein the ornamental plant cells are cells of a perennial plant.

34. The method of claim 33 wherein the ornamental plant cells are Petunia or Clematis cells.

35. The method of claim 20 wherein the ornamental plant cells are cells of a woody plant.

36. The method of claim 35 wherein the ornamental plant cells are cells of a poplar, apple or chokeberry.

37. The method of claim 23 wherein the ornamental plant cells are cells of a herbaceous plant.

38. The method of claim 23 wherein the expression cassette further comprises a transcription termination sequence.

39. The method of claim 38 wherein the transcription termination sequence is a 7S terminator sequence.

40. The method of claim 23 wherein flower longevity is increased at least 1.1-fold in the differentiated transformed female-sterile plant relative to the corresponding plant which lacks the expression cassette.

41. The method of claim 23 wherein flower longevity is increased at least 1.2-fold in the differentiated transformed female-sterile plant relative to the corresponding plant which lacks the expression cassette.

42. The method of claim 23 wherein flower longevity is increased at least 1.5-fold in the differentiated transformed female-sterile plant relative to the corresponding plant which lacks the expression cassette.

43. Progeny of the differentiated transformed female-sterile plant prepared by the method of claim 23.

44. A differentiated transformed female-sterile plant prepared by the method of claim 23.

45. A method to prepare a male-sterile, female-sterile plant having an increased number of flowers or flowers with increased longevity, comprising:
introducing to the plant of claim 21 or 22 a recombinant DNA molecule comprising an expression cassette comprising a pistil-specific promoter operably linked to DNA segment encoding a gene product, the expression of which in a cell results in ablation of that cell, so as to yield a male-sterile, female-sterile plant having an increased number of flowers or flowers with increased longevity.

46. A method to prepare a male-sterile, female-sterile plant having an increased number of flowers or flowers with increased longevity, comprising: crossing the plant of claim 21 or 22 with the plant of claim 43 or 44, so as to yield a male-sterile, female-sterile plant having an increased number of flowers or flowers with increased longevity.

47. A method to prepare a male-sterile, female-sterile plant having an increased number of flowers or flowers with increased longevity, comprising:
a) contacting cells of an ornamental plant with a first recombinant DNA molecule comprising a first expression cassette comprising a pistil-specific promoter operably linked to DNA segment encoding a gene product, the expression of which in a cell results in ablation of that cell, and a second recombinant DNA molecule comprising a second expression cassette comprising a stamen-specific promoter operably linked to DNA segment encoding a gene product, the expression of which in a cell results in ablation of that cell, so as to yield transformed plant cells; and
b) regenerating the transformed plant cells to provide a differentiated transformed male-sterile, female-sterile plant having an increase in the number of flowers or flower longevity, or both, relative to the number of flowers or flower longevity of a corresponding plant which does not comprise one or both expression cassettes.
